SEQUENCE LISTING (68 sequences)

SEQ ID NO: 1 - DNA, 414 bp, Bacillus subtilis
atgaaaaatagaatcgtatttttatatgcttcctgggttgtggctcttatcgctatgctgggcagcctgtatttcagtgaaatcagaaagtttattccatgtgaactgtgctggtaccagcgtatcctcatgtatccgctcgtcctgattttaggcatcgccacctttcaaggggacacacgagtgaaaaaatatgtgctcccgatggcgattattggggcattcatttcgatcatgcattacttagagcaaaaagtgcccggctttagcggcattaagccatgtgtcagcggcgtgccgtgctcgggccaatatattaactggtttggttttattacgattccattcctggccctgattgcttttatcctgattatcatttttatgtgcctgctgaaaggcgaaaaatctgaa

SEQ ID NO: 2 - protein, 138 aa, Bacillus subtilis
MKNRIVFLYASWVVALIAMLGSLYFSEIRKFIPCELCWYQRILMYPLVLILGIATFQGDTRVKKYVLPMAIIGAFISIMHYLEQKVPGFSGIKPCVSGVPCSGQYINWFGFITIPFLALIAFILIIIFMCLLKGEKSE

SEQ ID NO: 3 - DNA, 666 bp, Bacillus subtilis
gtgaaaaagaaacagcagtcttctgcgaaattcgcggtcatccttacggttgtggttgttgtattgctagcagccattgtcatcattaataacaaaacggaacaaggcaatgatgcggtgtccggacagccgtctatcaaagggcagcctgtgcttggcaaggacgatgcaccggtaactgtagtagaattcggagattacaaatgtccgtcttgcaaggtgtttaacagtgacatctttccaaaaatacaaaaagactttattgataagggcgatgtgaaattttctttcgtgaacgtcatgttccatggaaaaggttcaaggttagcggctcttgcatctgaagaagtatggaaggaagaccctgactctttctgggatttccatgaaaagctgttcgaaaaacagccggatacggaacaggaatgggtaaccccaggcctccttggtgatcttgcaaaaagcaccacaaagataaaacctgagacgctcaaggaaaatcttgataaggaaacattcgcttctcaagtggaaaaggattctgaccttaatcaaaaaatgaacatacaggcaacaccgacgatttacgtcaatgataaagtgatcaaaaattttgcggattatgatgaaatcaaagagacaatagaaaaagagctgaaagggaag

SEQ ID NO: 4 - protein, 222 aa, Bacillus subtilis
MKKKQQSSAKFAVILTVVVVVLLAAIVIINNKTEQGNDAVSGQPSIKGQPVLGKDDAPVTVVEFGDYKCPSCKVFNSDIFPKIQKDFIDKGDVKFSFVNVMFHGKGSRLAALASEEVWKEDPDSFWDFHEKLFEKQPDTEQEWVTPGLLGDLAKSTTKIKPETLKENLDKETFASQVEKDSDLNQKMNIQATPTIYVNDKVIKNFADYDEIKETIEKELKGK

SEQ ID NO: 5 - DNA, 25 bp, Bacillus subtilis
ggatccattatgtagggcgtaaagc

SEQ ID NO: 6 - DNA, 31 bp, Bacillus subtilis
ttagcaagcttaatcactttaatgccctcag

SEQ ID NO: 7 - DNA, 31 bp, Bacillus subtilis
tgattaagcttgctaatccgcaggacacttc

SEQ ID NO: 8 - DNA, 26 bp, Bacillus subtilis
ggtaccaacactgcctctctcatctc

SEQ ID NO: 9 - DNA, 5835 bp, Bacillus licheniformis
  misc_feature (2705)..(2705): n=a,c,g, or t
aattcagatctaaagataatatctttgaattgtaacscccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggccacattgaaaggggaggagaatcatgaaacaacaaaaacggctttacgcccgattgctgacgctgttatttgcgctcatcttcttgctgcctcattctgcagccgcggcacatcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttatgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtaaagcttgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttctatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacgataatgctagctatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagncgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgacgccaaaaaagctgtcctcccctcgttggggtcggacagctttttttatgcgatggaatggctgtcagccgatttttcatgcggccgcgtcgactagaagagcagagaggacggatttcctgaaggaaatccgtttttttattttgcccgtcttataaatttcgttgagataactagtataagatctataatcgataagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtg

SEQ ID NO: 10 - DNA, 40 bp, Bacillus licheniformis
gaattcacgcgtgttaactatgattaggagtgtttgcatt

SEQ ID NO: 11 - DNA, 40 bp, Bacillus licheniformis
gcggccgctatactagttatctcaacgaaatttataagac

SEQ ID NO: 12 - DNA, 1080 bp, Dictyoglomus thermophilum
atgtttcttaaaaaacttagtaagttgctcttagtcgtactacttgtcgcagtgtatacgcaagttaatgctcaaacgtctataacactaacaagtaatgcaagcggtacttttgatggctactactatgaactatggaaagatacagggaatacaaccatgactgtatacacacaaggaaggtttagctgtcagtggagcaatataaacaatgcattattcagaacaggtaagaagtacaaccaaaactggcagtcattaggcactattagaatcacctactcagccacatataatcctaatggtaactcctacttatgtatctatggttggtctactaatcctttagtagagttttacattgtagaaagttggggtaattggcgtccaccaggtgcaacctctcttggacaggttactatcgacggtggtacctatgacatttacagaactacccgtgtaaatcagccatctattgtcggtacagctacttttgatcaatattggagtgtaaggacatctaagagaacaagtggaacagtcactgtaacagatcactttagggcatgggcaaatagaggtttaaaccttggtactattgatcaaattactctttgtgttgaaggatatcaaagcagtggttcggctaatataacacaaaatactttttctcaaggtagcagtagtggaagtagtgggggcagtagtggtagtacaacaactactagaatagaatgtgaaaacatgtcattaagtgggccctatgtatcaagaattacaaatccatttaatggtatagctctttatgcaaatggagatactgcaagagctacagtaaacttcccagcaagtcgtaactataatttcaggttaagaggatgcggaaataacaataatttagctcgggttgatttacgaatagatgggaggactgtaggtacgttctattatcagggaacatatccttgggaggctcctatagacaatgtatacgtgagtgcaggttctcatacagtggaaattacagttacggctgataatgggacatgggatgtttatgcagattatctggtgatacag

SEQ ID NO: 13 - protein, 360 aa, Dictyoglomus thermophilum
MFLKKLSKLLLVVLLVAVYTQVNAQTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGSSSGSSGGSSGSTTTTRIECENMSLSGPYVSRITNPFNGIALYANGDTARATVNFPASRNYNFRLRGCGNNNNLARVDLRIDGRTVGTFYYQGTYPWEAPIDNVYVSAGSHTVEITVTADNGTWDVYADYLVIQ

SEQ ID NO: 14 - DNA, 668 bp, Dictyoglomus thermophilum
cttttagttcatcgatcgcatcggctgctcagacatcaatcacacttacatctaacgcatcaggcacattcgacggctattactacgagctttggaaggacacaggcaacacgactatgactgtatacactcaaggtcgcttctcatgccagtggtctaacatcaacaacgcgcttttccgcacgggcaagaagtacaaccagaactggcaatctcttggcactatccgcatcacttattctgcgacatacaacccgaacggcaactcttacctttgtatctacggctggtctacgaacccgcttgttgagttctacatcgtagagtcttggggcaactggcgtcctcctggcgcaacatctcttggccaggttacaatcgatggtggcacatatgacatctaccgcactactcgcgttaaccagcctagcatcgttggcacagctactttcgaccaatactggagcgttcgcactagcaagcgcacatctggcacagttacggttacggaccactttcgcgcatgggcaaatcgtggccttaaccttggcacaatcgaccaaatcacactttgtgttgagggctaccagtcttctggcagcgcaaacatcactcaaaacactttctctcagggcagccatcagcaccaacaccagcatcaaccctag

SEQ ID NO: 15 - protein, 221 aa, Dictyoglomus thermophilum
FSSSIASAAQTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGSHQHQHQHQP

SEQ ID NO: 16 - DNA, 48 bp, Dictyoglomus thermophilum
cttttagttcatcgatcgcatcggctgctcagacatcaatcacactta

SEQ ID NO: 17 - DNA, 49 bp, Dictyoglomus thermophilum
ctagggttgatgctggtgttggtgctgatggctgccctgagagaaagtg

SEQ ID NO: 18 - DNA, 21 bp, Dictyoglomus thermophilum
gagtatcgccagtaaggggcg

SEQ ID NO: 19 - DNA, 22 bp, Dictyoglomus thermophilum
agccgatgcgatcgatgaacta

SEQ ID NO: 20 - DNA, 53 bp, Dictyoglomus thermophilum
catcagcaccaacaccagcaccagccataatcgcatgttcaatccgctccata

SEQ ID NO: 21 - DNA, 23 bp, Dictyoglomus thermophilum
gcagccctaaaatcgcataaagc

SEQ ID NO: 22 - DNA, 40 bp, Dictyoglomus thermophilum
gagctctataaaaatgaggagggaaccgaatgaagaaacc

SEQ ID NO: 23 - DNA, 28 bp, Dictyoglomus thermophilum
acgcgtttagctgccctgagagaaagtg

SEQ ID NO: 24 - DNA, 20 bp, Dictyoglomus thermophilum
ccgttggggaaaattgtcgc

SEQ ID NO: 25 - DNA, 20 bp, Dictyoglomus thermophilum
gcgacaattttccccaacgg

SEQ ID NO: 26 - DNA, 693 bp, Dictyoglomus thermophilum
atgaagaaaccgttggggaaaattgtcgcaagcaccgcactactcatttctgttgcttttagttcatcgatcgcatcggctgctcagacatcaatcacacttacatctaacgcatcaggcacattcgacggctattactacgagctttggaaggacacaggcaacacgactatgactgtatacactcaaggtcgcttctcatgccagtggtctaacatcaacaacgcgcttttccgcacgggcaagaagtacaaccagaactggcaatctcttggcactatccgcatcacttattctgcgacatacaacccgaacggcaactcttacctttgtatctacggctggtctacgaacccgcttgttgagttctacatcgtagagtcttggggcaactggcgtcctcctggcgcaacatctcttggccaggttacaatcgatggtggcacatatgacatctaccgcactactcgcgttaaccagcctagcatcgttggcacagctactttcgaccaatactggagcgttcgcactagcaagcgcacatctggcacagttacggttacggaccactttcgcgcatgggcaaatcgtggccttaaccttggcacaatcgaccaaatcacactttgtgttgagggctaccagtcttctggcagcgcaaacatcactcaaaacactttctctcagggcagc

SEQ ID NO: 27 - protein, 231 aa, Dictyoglomus thermophilum
MKKPLGKIVASTALLISVAFSSSIASAAQTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGS

SEQ ID NO: 28 - DNA, 20 bp, Bacillus subtilis
tatcaattggtaactgtatc

SEQ ID NO: 29 - DNA, 54 bp, Bacillus subtilis
cggcaaactgacaaataacagcgtgcacataagcaccaatcgggcgtaaagccg

SEQ ID NO: 30 - DNA, 59 bp, Dictyoglomus thermophilum
ctgttatttgtcagtttgccgattacaaaaacatcagcccagacatcaatcacacttac

SEQ ID NO: 31 - DNA, 20 bp, Dictyoglomus thermophilum
cctttgcggctttttgcatc

SEQ ID NO: 32 - DNA, 20 bp, Dictyoglomus thermophilum
cagattacaaatatattcgg

SEQ ID NO: 33 - DNA, 19 bp, Dictyoglomus thermophilum
gtagatgtcatatgtgcca

SEQ ID NO: 34 - DNA, 19 bp, Dictyoglomus thermophilum
tggcacatatgacatctac

SEQ ID NO: 35 - DNA, 26 bp, Bacillus subtilis
ggatccgcgatgggaggccttggctc

SEQ ID NO: 36 - DNA, 26 bp, Bacillus subtilis
cccgggttcactccgacacctcatcg

SEQ ID NO: 37 - DNA, 46 bp, Bacillus subtilis
cgatgaggtgtcggagtgaacccgggtatgtgcctgctgaaaggcg

SEQ ID NO: 38 - DNA, 26 bp, Bacillus subtilis
ggtacctccctgctcccatagcgctg

SEQ ID NO: 39 - DNA, 411 bp, Bacillus subtilis
atgaaaaagtggattgttttatttcttgttttaatagcagcagccattagtattttcgtttatgtttctacaggtagcgaaaaacctttttataatgatataaatttaactcaatatcaaaaagaagtagactctaaaaaacctaaatttatttatgtttatgagacaagttgtcctccttgtcaagaaataaaacctgagttaaatgaagtaattaaaaaagaaaagttaaaagtacaggctttaaatattgaagaaaaggaaaattataacactgaatttttagataaatataatttgaataaaactccaacgattctctattacaaagatggcaaagaaaaagatcggttagagggctatagaagtgcaagccaaatagaaaagttctttgataaaaatggtgataga

SEQ ID NO: 40 - protein, 137 aa, Bacillus subtilis
MKKWIVLFLVLIAAAISIFVYVSTGSEKPFYNDINLTQYQKEVDSKKPKFIYVYETSCPPCQEIKPELNEVIKKEKLKVQALNIEEKENYNTEFLDKYNLNKTPTILYYKDGKEKDRLEGYRSASQIEKFFDKNGDR

SEQ ID NO: 41 - DNA, 444 bp, Bacillus subtilis
atgaatacaagatatgtaaaatcattttttttattactgttttttctctctttctttggcacaatggctagtttattctacagtgagatcatgcatttcaaaccatgtgttctatgttggtatcaaagaatatttctatatcctatacctattatcttactaataggcttattaaaaaaagatcttaattcgatattttatgttgttttcctttcatcaattggattgattattgcgttttatcattatattatccaacttacacaaagcaaaagtgtcgtatgtgaaattggaaccaacagctgcgcaaaaattgaagtagagtatctaggctttattacattacccttaatgagttcagtatgttttgcattgatatttggtataggactgaaattaattatcaaaagcaagaaattaaaacaaaatcaacatgtatataat

SEQ ID NO: 42 - protein, 148 aa, Bacillus subtilis
MNTRYVKSFFLLLFFLSFFGTMASLFYSEIMHFKPCVLCWYQRIFLYPIPIILLIGLLKKDLNSIFYVVFLSSIGLIIAFYHYIIQLTQSKSVVCEIGTNSCAKIEVEYLGFITLPLMSSVCFALIFGIGLKLIIKSKKLKQNQHVYN

SEQ ID NO: 43 - DNA, 24 bp, Bacillus subtilis
caatggattcgcaggtattagatg

SEQ ID NO: 44 - DNA, 48 bp, Bacillus subtilis
catcctttcacaatttgtctacagctgagcttttcctaatccactacc

SEQ ID NO: 45 - DNA, 48 bp, Bacillus subtilis
ggtagtggattaggaaaagctcagctgtagacaaattgtgaaaggatg

SEQ ID NO: 46 - DNA, 48 bp, Bacillus subtilis
ccctcttacaaggcgggttacttccaagtgttcgcttcgctctcactg

SEQ ID NO: 47 - DNA, 48 bp, Bacillus subtilis
cagtgagagcgaagcgaacacttggaagtaacccgccttgtaagaggg

SEQ ID NO: 48 - DNA, 24 bp, Bacillus subtilis
aagacgagtgtcgggtaacgtagg

SEQ ID NO: 49 - DNA, 420 bp, Bacillus amyloliquefaciens
atgaaaaatagaatcgtatttttatatgcttcttgggtgatagctcttgctgccatgctcggcagcctatatttcagcgagatcagaaaattcataccgtgcgagctgtgctggtatcaaagaattctcatgtacccgcttgtcctgattttaggaatcgccaccttccagggtgacacgcgcgtgaaaaaatatgtgctgccgatggccgtcatcggcgcgtttatatcgatcatgcattaccttgagcagaaggtgcccggtttcagcggcatcaagccgtgtgtctacggcgtgccttgctcatctgaatatattaactggttcggtttcattaccattcctttcctggcattgacggcatttattctcattattatctgcatgtgttttgtcaaaacggaacgcgcggcaaaataa

SEQ ID NO: 50 - protein, 139 aa, Bacillus amyloliquefaciens
MKNRIVFLYASWVIALAAMLGSLYFSEIRKFIPCELCWYQRILMYPLVLILGIATFQGDTRVKKYVLPMAVIGAFISIMHYLEQKVPGFSGIKPCVYGVPCSSEYINWFGFITIPFLALTAFILIIICMCFVKTERAAK

SEQ ID NO: 51 - DNA, 414 bp, Bacillus licheniformis
atgaaaaataaactgctttttctgtacggcgcctggatcgtctcattaacggcgacgctaggcagcttgtacttcagcgaaatccgcaaatttattccttgcgaactgtgctggtatcagcggattatgatgtatccgctcgtgctgattctcggaattgcgacatttcagggcgatgcccgcgtgaaaaaatacgtgctgccgatggcggtgatcggcgcaggcatttccctgatgcactacatggaacaaaaaattcccggattcaacggcattaaaccgtgtgtcacaggagtgccttgctcagggcagtatatcaattggttcggtttcatcacgattccgtttctcgcccttattgcatttattttgattatcatttttatgtgctttctcaaggggaaagacgagtaa

SEQ ID NO: 52 - protein, 137 aa, Bacillus licheniformis
MKNKLLFLYGAWIVSLTATLGSLYFSEIRKFIPCELCWYQRIMMYPLVLILGIATFQGDARVKKYVLPMAVIGAGISLMHYMEQKIPGFNGIKPCVTGVPCSGQYINWFGFITIPFLALIAFILIIIFMCFLKGKDE

SEQ ID NO: 53 - DNA, 414 bp, Bacillus pumilus
atgaagaataagcttatttacttgtacagtgcttggattgtctcgatcgttgcgacgatgagcagcctgtatttaagtgaaattaaaaagtttattccatgcgatatgtgctggttccagcgcattttcatgtacccgctcgtgcttttactaggaattgctacgttccggggtgatgttaaagtgaagtattacgtactgcctttggctgtcatcggtgcttgcttttccatctatcattatatggaacaaaaaatcccaggctttgcgtcgattcgcccttgtctcagcggcattccttgttctgtagattatttgaactggtttggttttatcaccattccgctattagcacttattgcatttattctgatcatcatcagtatgctgctgttaaatgcaaaagaagattaa

SEQ ID NO: 54 - protein, 137 aa, Bacillus pumilus
MKNKLIYLYSAWIVSIVATMSSLYLSEIKKFIPCDMCWFQRIFMYPLVLLLGIATFRGDVKVKYYVLPLAVIGACFSIYHYMEQKIPGFASIRPCLSGIPCSVDYLNWFGFITIPLLALIAFILIIISMLLLNAKED

SEQ ID NO: 55 - DNA, 420 bp, Bacillus cereus
atgggacgagaaaaaaagcaagaatatgctttatttaccgcgtggggagcttcttttattgctacactagggagtctatacttttccgaaatcatgaaatttgagccttgtgtcctttgttggtatcaacgtatttttatgtatccattcgttttatggctcggtatcgctgtagtaaaaaaagactatcgcatcgcaaattattctttaccaatcgcaagtatcggtgcttgtatttctttatatcactatgcaattcaaaagatcgcagcattttcagctgccggggcagcttgcggccgtgtaccatgtacgggagaatacataaactggttcggctttgtgacaatcccgtttttagcacttatcggctttattacaatcgctgtttgtagctttattgtcattaaaaacaaataa

SEQ ID NO: 56 - protein, 139 aa, Bacillus cereus
MGREKKQEYALFTAWGASFIATLGSLYFSEIMKFEPCVLCWYQRIFMYPFVLWLGIAVVKKDYRIANYSLPIASIGACISLYHYAIQKIAAFSAAGAACGRVPCTGEYINWFGFVTIPFLALIGFITIAVCSFIVIKNK

SEQ ID NO: 57 - DNA, 414 bp, Bacillus halodurans
atgagcaaaaaggttgaaaacctcatgcttggctcttggcttacggcattaacggcgatgcttggctcgctttatttttctgaaattaggatgtacgagccttgtaccctgtgttggtaccagcgcatcatcatgtatccgctcgtactcattctttttattggctatcttaaacgggatgtcaacgtggcgttgtactccctctggttttcgctcatcgggatgttcacgtcgctctatcattattcaatacaaaagctcccatttttaacggatgccgctcccgcttgtggtcgggttccgtgtacaggtcagtatatcaattggttcggctttgtgacgattccatttttagcttttacagcatttgtcattattttcatttgcagtttacttattattcgtgagaagtag

SEQ ID NO: 58 - protein, 137 aa, Bacillus halodurans
MSKKVENLMLGSWLTALTAMLGSLYFSEIRMYEPCTLCWYQRIIMYPLVLILFIGYLKRDVNVALYSLWFSLIGMFTSLYHYSIQKLPFLTDAAPACGRVPCTGQYINWFGFVTIPFLAFTAFVIIFICSLLIIREK

SEQ ID NO: 59 - DNA, 414 bp, Bacillus clausii
gtgaaaaaacaagttgagaacgggcttttatttgcatgggtgacagctttagttgcaacgcttggctcattgtatttctcagaaattcgccagtttgaaccttgtgctctttgctggtatcaacgaattttaatgtacccgctagttgtccttttggcgataggcatcattcgcaaagattcaactgcagcgatttattcagctgttcttgccggcattggcttctgcatatccgcttatcattattcaattcagaaacttcccgtgcaagaaggcaatgtgttagggtgcggggctgtcccatgtacaggagaatatattaattggcttggctttataacgattccttttcttgctggaattgcattcttaatgatttttttaacaagtatgtatatcattcgaaaccgctag

SEQ ID NO: 60 - protein, 137 aa, Bacillus clausii
MKKQVENGLLFAWVTALVATLGSLYFSEIRQFEPCALCWYQRILMYPLVVLLAIGIIRKDSTAAIYSAVLAGIGFCISAYHYSIQKLPVQEGNVLGCGAVPCTGEYINWLGFITIPFLAGIAFLMIFLTSMYIIRNR

SEQ ID NO: 61 - DNA, 672 bp, Bacillus amyloliquefaciens
gtgaaaaagaaacaatcgtctgcaaaatttgcggtgattctgacgttaatcgttgtcgttttatttgcggccatcgtaattattaacaaccaaacggaaaaagcgggcgaaacagtcgccgaacagccttccattaaaggacagcccgtgctcggcaaagacagcgcccctgttacggtagttgaattcggagattacaagtgtccgtcatgcaaagtatttaacagcgatatctttccgaaaatcaaaaaagattttatcgataaaggcgacgtgaaattttcatttgtgaacgttatgtaccacggaagcggctcgcgtctggcggctcttgcttcagaagaagtgtggaaagaagacccggcatcattctgggcattccatgaaaagctgtttgaacagcagccgtcaagcgaacaggaatgggtcacgcctgcattgcttgaaaaaacggtcaaaagcaccgcgaaaaaggttgatcctgacaagctgaaggaaaaccttgataaagagacatttgcaaaagagctgaaagctgacactgacctgaatgataaattaaacattacggcgacaccgacgatttatgtaaatgacaaagtgattaatgacttctctaaatatgatgagatttcaaaaacgattaagaaagagctgaaaaatgaaaaatag

SEQ ID NO: 62 - protein, 223 aa, Bacillus amyloliquefaciens
MKKKQSSAKFAVILTLIVVVLFAAIVIINNQTEKAGETVAEQPSIKGQPVLGKDSAPVTVVEFGDYKCPSCKVFNSDIFPKIKKDFIDKGDVKFSFVNVMYHGSGSRLAALASEEVWKEDPASFWAFHEKLFEQQPSSEQEWVTPALLEKTVKSTAKKVDPDKLKENLDKETFAKELKADTDLNDKLNITATPTIYVNDKVINDFSKYDEISKTIKKELKNEK

SEQ ID NO: 63 - DNA, 696 bp, Bacillus licheniformis
gtgaagaagaaacagcagtcaccgatgaaatttgcagtgattatgacagtcgtggtcgtttttctgatcggcgcacttgtcgtaatcaacaatcaaacccaaaatgcttcgcaaacctttgatgacaagccttcaactgaaggacagccgcttctaggcaacaaagatgcggctgtaacgatcacggaattcggagattacaaatgtcccagctgcaaacagtggactgagaccgtctttccggatttgaaaaaggattacatcgataaagatcaagttaatttttcatatattaacttcgtcaatgaacagcacggcagaggctctgaattgagcgccctcgcttccgagcaggtatggaaggaagatccggattcattctggaagttccatgaggcgttgtacaaggcgcagcctgacaatgacacgatggaaaacgagtgggcgacgccggcaaaattggcggacatcacggaagccaatacgaaaatcaaacgcgataagcttgtcagcagcttaaatgacaaaacgttcgctgagcaattaaaaacggacaattcgctcatcaacaaatacggtgtagactcgacgccgacgatctttgtcaacggcgtaaaaatcgacaaaccgtttgattatgacaaaatcaaagaaacgatcgagaaagagctgaaaggccagtctgatgaaaaataa

SEQ ID NO: 64 - protein, 231 aa, Bacillus licheniformis
MKKKQQSPMKFAVIMTVVVVFLIGALVVINNQTQNASQTFDDKPSTEGQPLLGNKDAAVTITEFGDYKCPSCKQWTETVFPDLKKDYIDKDQVNFSYINFVNEQHGRGSELSALASEQVWKEDPDSFWKFHEALYKAQPDNDTMENEWATPAKLADITEANTKIKRDKLVSSLNDKTFAEQLKTDNSLINKYGVDSTPTIFVNGVKIDKPFDYDKIKETIEKELKGQSDEK

SEQ ID NO: 65 - DNA, 687 bp, Bacillus pumilus
gtgagtaagaaaaataatcaatcttcatccattaaatttgctgtcattttaaccattattgccgctcttctcatcggtatatttgttgtgattggaaacaagaacagccaagaagcacaaacagttgacagtaagccttcgattcaaggacaacctgtcataggagacaaaaacgcagcagtgcaaattgtcgagtttggagactacaaatgtccgtcatgtaaatcatttgaaacagacattttcccaaaactgaaagcagactacatagataaaggcgatgtatccttctcatttattaacttaccactgcctgttcatggagatggcgcagtgttagcagcactagcttctgaagaagtgtggaaagaagatccaaaaaacttctgggcattccatgaagctgtctatcaagcacagccagatagtgaagcagaatgggtgacgcctgctaagctgactgaactggcgaaaaagacaacaaaaattgatacggacaaactcaaagatcatttatcaaagaaaacgtatcagccgcagctgaacacagacaatcagcttgtgaacaaatacaaagtgaattcaacaccaacgatctttattaacaataaacaagttcaaaatttctatgactatgatgaaatcaaagaattaattgatcaagaacttaaagggaagaaatcatga

SEQ ID NO: 66 - protein, 228 aa, Bacillus pumilus
MSKKNNQSSSIKFAVILTIIAALLIGIFVVIGNKNSQEAQTVDSKPSIQGQPVIGDKNAAVQIVEFGDYKCPSCKSFETDIFPKLKADYIDKGDVSFSFINLPLPVHGDGAVLAALASEEVWKEDPKNFWAFHEAVYQAQPDSEAEWVTPAKLTELAKKTTKIDTDKLKDHLSKKTYQPQLNTDNQLVNKYKVNSTPTIFINNKQVQNFYDYDEIKELIDQELKGKKS

SEQ ID NO: 67 - DNA, 654 bp, Bacillus cereus
atgaaatcatcgaacaaactcatggctcttggtatagttttttccattgcagtattgattgtgatcggaacgattgcgtatagcatcataaatgacaaaaaagataaagggaatgagatgtttgcttattccacacaacaatctttagggaaagatgatgctccggttaaggtagttgaatttggagacttcaaatgtcccgcttgtcgtacttgggatgtaacagtattgccacgattaaaagaagagtatattgataaaggtaaagtgcaattatactttattaattttccgtttattgggaaagactctgatttaggtgcagcagctggtgaagcaatttataaacaagataaagattcattctggactttctatgatgagatttatcaaagtcaaaagaaagatacggaagaatggattacagaagaattacttcttaacattgtgaaagagaagcttccgaaagttgatgtagatcaatttaagaaagatttacacagtaaagaaataaaagaaaaagtacgtaaagattcagatcgtgctcaaaaattaaaagttcaaggtgctccttcagtatatataaacggaaatcttgcaaatcctgatttcgatagtatgaagaaggcgattgataaagaattgaaaaagtga

SEQ ID NO: 68 - protein, 217 aa, Bacillus cereus
MKSSNKLMALGIVFSIAVLIVIGTIAYSIINDKKDKGNEMFAYSTQQSLGKDDAPVKVVEFGDFKCPACRTWDVTVLPRLKEEYIDKGKVQLYFINFPFIGKDSDLGAAAGEAIYKQDKDSFWTFYDEIYQSQKKDTEEWITEELLLNIVKEKLPKVDVDQFKKDLHSKEIKEKVRKDSDRAQKLKVQGAPSVYINGNLANPDFDSMKKAIDKELKK

(19) United States
(12) Patent Application Publication
(10) Pub. No.: US 2012/0329090 A1
(43) Pub. Date: Dec. 27, 2012

(54) Methods for Producing Heterologous Polypeptides in Thiol-Disulfide Oxidoreductase-Deficient Bacterial Mutant Cells

(75) Inventors: William Widner, Davis, CA (US); Bjarke Christensen, Lyngby (DK)

(73) Assignees: NOVOZYMES A/S, Bagsvaerd (DK); NOVOZYMES, INC., Davis, CA (US)

(21) Appl. No.: 13/516,475

(22) PCT Filed: Dec. 17, 2010
(86) PCT No.: PCT/US10/61068

§ 371 Date: Sep. 11, 2012

Related U.S. Application Data
(60) Provisional application No. 61/288,516, filed on Dec. 21, 2009.

(51) Int. Cl. C12N 1/21; C12N 15/87; C12P 21/00

(52) U.S. Cl. 435/69.1; 435/252.31; 435/471

(57) ABSTRACT

The present invention relates to methods of producing a heterologous polypeptide, comprising: (a) cultivating a mutant of a parent bacterial cell in a medium for the production of the heterologous polypeptide, wherein (i) the mutant cell comprises a first polynucleotide encoding the heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, and (ii) the mutant cell is deficient in production of the thiol-disulfide oxidoreductase compared to the parent bacterial cell when cultivated under the same conditions; and (b) recovering the heterologous polypeptide from the cultivation medium. The present invention also relates to such bacterial mutants and methods for producing such bacterial mutants.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to thiol-disulfide oxidoreductase-deficient bacterial mutant cells and methods of producing heterologous polypeptides in such thiol-disulfide oxidoreductase-deficient bacterial mutant cells.

2. Description of the Related Art

The folding of polypeptide chains depends upon chaperones and folding catalysts, such as thiol-disulfide oxidoreductases. Thiol-disulfide oxidoreductases catalyze thiol/disulfide interchange reactions and promote disulfide formation, isomerization or reduction, thereby facilitating formation of correct disulfide pairings (Hart et al., 1995, Current Opinion in Structural Biology 5: 92-102). Such oxidoreductases interact directly with newly synthesized secretory proteins and are required for the folding of nascent polypeptides in the endoplasmic reticulum (ER) of eukaryotic cells.

Bacilli are well established industrially as host cell systems for the recombinant production of heterologous proteins as a result of their ability to express and secrete their products. However, Bacillus host cells with the desirable traits of protein expression and secretion may not necessarily have the most desirable characteristics for the production of biologically active heterologous proteins. The presence of a thiol-disulfide oxidoreductase native to the host cell may catalyze the formation of incorrect disulfide pairings in a heterologous protein.

Meima et al., 2002, Journal of Biological Chemistry 277: 6994-7001, disclose the bdbCD operon of Bacillus subtilis encoding thiol-disulfide oxidoreductases required for competence development. Erlendsson and Hederstedt, 2002, Journal of Bacteriology 184: 1423-1429, disclose that mutations in the thiol-disulfide oxidoreductases BdbC and BdbD can suppress cytochrome c deficiency of CcdA-defective Bacillus subtilis cells.

U.S. Pat. Nos. 6,521,421 and 7,037,714 disclose expression vectors encoding Bacillus subtilis disulfide bond isomerase and methods of secreting proteins in gram-positive microorganisms using the same.

The present invention provides improved bacterial host cells deficient in the production of thiol-disulfide oxidoreductase for the production of heterologous proteins and methods of producing heterologous polypeptides in such thiol-disulfide oxidoreductase-deficient bacterial mutant cells.

### SUMMARY OF THE INVENTION

The present invention relates to isolated mutants of a parent bacterial cell, comprising a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, wherein the mutant cell is deficient in production of the thiol-disulfide oxidoreductase compared to the parent bacterial cell when cultivated under the same conditions.

The present invention also relates to methods of producing a heterologous polypeptide, comprising:

(a) cultivating a mutant of a parent bacterial cell in a medium for the production of the heterologous polypeptide, wherein (i) the mutant cell comprises a first polynucleotide encoding the heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, and (ii) the mutant cell is deficient in production of the thiol-disulfide oxidoreductase compared to the parent bacterial cell when cultivated under the same conditions; and

(b) recovering the heterologous polypeptide from the cultivation medium.

The present invention further relates to methods of obtaining a mutant of a parent bacterial cell, comprising:

(a) introducing into the parent bacterial cell a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide; and

(b) identifying the mutant cell from step (a) comprising the modified polynucleotide, wherein the mutant cell is deficient in the production of the thiol-disulfide oxidoreductase.

### BRIEF DESCRIPTION OF THE FIGURES

FIG. 1 shows a restriction map of pNNB194-ispAΔ.

FIG. 2 shows a restriction map of pMOL2657.

FIG. 3 shows a restriction map of pRB217.

FIG. 4 shows a restriction map of pRB219.

FIG. 5 shows a restriction map of pSMO280.

FIG. 6 shows a restriction map of pIC20R-amyL.

FIG. 7 shows a restriction map of pHP13 ampMCS-amyL.

FIG. 8 shows a restriction map of pSJ2882-amyL orf.

FIG. 9 shows a restriction map of pMRT135.

FIG. 10 shows a restriction map of pBW223.

FIG. 11 shows a restriction map of pBW224.

FIG. 12 shows a restriction map of pBW226.

### DEFINITIONS

Thiol-disulfide oxidoreductase: The term “thiol-disulfide oxidoreductase” means an enzyme that catalyzes oxidoreductase reactions by a dithiol/disulfide exchange mechanism involving two redox-active cysteines interchange reactions, which promote disulfide formation, isomerization or reduction, thereby facilitating the formation of correct disulfide pairings (Ortenberh and Beckwith, 2003, Antioxidants & Redox Signaling 5: 403-11; Meyer et al., 2009, Annu. Rev. Genet. 2009. 43: 335-367). For purposes of the present invention, thiol-disulfide oxidoreductase activity is determined according to the procedure described by Holmgren, 1979, Journal of Biological Chemistry 254: 9627-9632 or any other assay well known in the art.

Isolated or Purified: The term “isolated” or “purified” means a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, or at least 95% pure, as determined by SDS-PAGE, and a polynucleotide may be at least 1% pure, e.g., at least 5% pure, at least 10% pure, at least 20% pure, at least 40% pure, at least 60% pure, at least 80% pure, at least 90% pure, or at least 95% pure, as determined by agarose electrophoresis.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide.

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Homologous sequence: The term “homologous sequence” means a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with the Bacillus subtilis thiol-disulfide oxidoreductase of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

Fragment: The term “fragment” means a polypeptide having one or more (several) amino acids deleted from the amino and/or carboxyl terminus of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof; wherein the fragment has thiol-disulfide oxidoreductase activity.

Subsequence: The term “subsequence” means a polynucleotide having one or more (several) nucleotides deleted from the 5′ and/or 3′ end of the polypeptide coding sequence of SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof; wherein the subsequence encodes a polypeptide fragment having thiol-disulfide oxidoreductase activity.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, synthetic, or recombinant polynucleotide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence.

Control sequences: The term “control sequences” means all components necessary for the expression of a polynucleotide encoding a polypeptide of interest. Each control sequence may be native or foreign to the polynucleotide encoding the polypeptide or native or foreign to each other. Such control sequences include, but are not limited to, a propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Host cell: The term “host cell” means any cell type that is susceptible to transformation, transfection, transduction, and the like with a nucleic acid construct or expression vector comprising a polynucleotide. The term “host cell” encompasses any progeny of a parent cell that is not identical to the parent cell due to mutations that occur during replication.

Introduction: The term “introduction” or variations thereof means the transfer of a DNA into a bacterial cell. The introduction of a DNA into a bacterial cell can be accomplished by any method known in the art, including, but not limited to, transformation, transfection, transduction, conjugation, and the like.

Transformation: The term “transformation” means introducing a purified DNA into a bacterial cell so that the DNA is maintained as a chromosomal integrant or as a self-replicating extra-chromosomal vector. The term “transformation” shall be generally understood to include transfection, transduction, conjugation, and the like.

Transfection: The term “transfection” means the transformation of a bacterial cell with a viral nucleic acid.

Transduction: The term “transduction” means the packaging of DNA from a first bacterial cell into a virus particle and the transfer of that bacterial DNA to a second bacterial cell by infection of the second cell with the virus particle.

Conjugation: The term “conjugation” means the transfer of DNA directly from one bacterial cell to another bacterial cell through cell-to-cell contact.

Transformant: The term “transformant” means any bacterial cell into which a DNA has been introduced. Consequently, the term “transformant” included transfectants, conjugants, and the like.

Donor Cell: The term “donor cell” means a cell that is the source of DNA introduced by any means to another cell.

Recipient cell: The term “recipient cell” means a cell into which DNA is introduced.

Modification: The term “modification” means introduction, substitution, or removal of one or more (several) nucleotides in a thiol-disulfide oxidoreductase gene, or a regulatory element required for the transcription or translation thereof; a gene disruption; a gene conversion; a gene deletion; or random or specific mutagenesis of a thiol-disulfide oxidoreductase gene. The deletion of the thiol-disulfide oxidoreductase gene may be partial or complete.

Deficient in the production of a thiol-disulfide oxidoreductase: The phrase “deficient in the production of a thiol-disulfide oxidoreductase” means a bacterial mutant cell which produces no detectable thiol-disulfide oxidoreductase encoded by a particular gene, or, in the alternative, produces preferably at least about 25% less, more preferably at least about 50% less, even more preferably at least about 75% less, and most preferably at least about 95% less thiol-disulfide oxidoreductase encoded by a particular gene compared to the parent bacterial cell when cultivated under the same conditions. The level of a thiol-disulfide oxidoreductase produced by a bacterial mutant cell of the present invention may be determined using methods well known in the art (see, for example, Holmgren, 1979, supra).

As used herein and in the appended claims, the singular forms “a,” “or,” and “the” include plural referents unless the context clearly dictates otherwise.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to isolated mutants of a parent bacterial cell, comprising a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, wherein the mutant cell is deficient in production of the thiol-disulfide oxidoreductase compared to the parent bacterial cell when cultivated under the same conditions.

The present invention also relates to methods of producing a heterologous polypeptide, comprising: (a) cultivating a mutant of a parent bacterial cell in a medium for the production of the heterologous polypeptide, wherein (i) the mutant cell comprises a first polynucleotide encoding the heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, and (ii) the mutant cell is deficient in production of the thiol-disulfide oxidoreductase compared to the parent bacterial cell when cultivated under the same conditions; and (b) recovering the heterologous polypeptide from the cultivation medium.

The present invention further relates to methods of obtaining a mutant of a parent bacterial cell, comprising: (a) introducing into the parent bacterial cell a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide; and (b) identifying the mutant cell from step (a) comprising the modified polynucleotide, wherein the mutant cell is deficient in the production of the thiol-disulfide oxidoreductase.

An advantage of the present invention is the elimination or reduction of a thiol-disulfide oxidoreductase(s) that can adversely affect production of a heterologous polypeptide by incorrectly catalyzing the formation of one or more (several) disulfide bonds between two or more (several) cysteines of the heterologous polypeptide resulting in the polypeptide having no or less biological activity. The deficiency in the production of the thiol-disulfide oxidoreductase prevents the formation of one or more (several) disulfide bonds between the two or more (several) cysteines of the heterologous polypeptide.

The bacterial mutant cells are cultivated in a nutrient medium suitable for production of a heterologous polypeptide of interest using methods known in the art. For example, the mutant cell may be cultivated by shake flask cultivation, or small-scale or large-scale fermentation (including continuous, batch, fed-batch, or solid state fermentations) in laboratory or industrial fermentors in a suitable medium and under conditions allowing the polypeptide of interest to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The polypeptide of interest can be recovered directly from the medium or the bacterial mutant cells.

The heterologous polypeptide of interest may be detected using methods known in the art that are specific for the polypeptide. These detection methods may include, for example, use of specific antibodies, high performance liquid chromatography, capillary chromatography, formation of an enzyme product, disappearance of an enzyme substrate, or SDS-PAGE. For example, an enzyme assay may be used to determine the activity of an enzyme. Procedures for determining enzyme activity are known in the art for many enzymes (see, for example, D. Schomburg and M. Salzmann (eds.), Enzyme Handbook, Springer-Verlag, New York, 1990).

The resulting polypeptide may be isolated using methods known in the art. For example, a polypeptide of interest may be isolated from the cultivation medium by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989).

### Parent Bacterial Cells

The parent bacterial cell may be any Gram-positive bacterium or any Gram-negative bacterium. Gram-positive bacteria include, but are not limited to, Bacillus, Streptococcus, Streptomyces, Staphylococcus, Enterococcus, Lactobacillus, Lactococcus, Clostridium, Geobacillus, and Oceanobacillus cells. Gram-negative bacteria include, but are not limited to, E. coli, Pseudomonas, Salmonella, Campylobacter, Helicobacter, Flavobacterium, Fusobacterium, Ilyobacter, Neisseria, and Ureaplasma cells. In the methods of the present invention, the parent bacterial cell may be a wild-type bacterial cell or a mutant thereof.

In the methods of the present invention, the parent bacterial cell may be any Bacillus cell. Bacillus cells useful in the practice of the present invention include, but are not limited to, Bacillus alkalophilus, Bacillus amyloliquefaciens, Bacillus brevis, Bacillus cereus, Bacillus circulans, Bacillus clausii, Bacillus coagulans, Bacillus firmus, Bacillus halodurans, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In one aspect, the parent Bacillus cell is a Bacillus amyloliquefaciens cell. In another aspect, the parent Bacillus cell is a Bacillus cereus. In another aspect, the parent Bacillus cell is a Bacillus clausii cell. In another aspect, the parent Bacillus cell is a Bacillus halodurans. In another aspect, the parent Bacillus cell is a Bacillus lentus cell. In another aspect, the parent Bacillus cell is a Bacillus licheniformis cell. In another aspect, the parent Bacillus cell is a Bacillus pumilus cell. In another aspect, the parent Bacillus cell is a Bacillus stearothermophilus cell. In another aspect, the parent Bacillus cell is a Bacillus subtilis cell.

In the methods of the present invention, the parent bacterial cell may be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus cells. In one aspect, the parent bacterial cell is a Streptococcus equisimilis cell. In another aspect, the parent bacterial cell is a Streptococcus pyogenes cell. In another aspect, the parent bacterial cell is a Streptococcus uberis cell. In another aspect, the parent bacterial cell is a Streptococcus equi subsp. Zooepidemicus cell.

In the methods of the present invention, the parent bacterial cell may be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans cells.

In one aspect, the parent bacterial cell is a Streptomyces achromogenes cell. In another aspect, the parent bacterial cell is a Streptomyces avermitilis cell. In another aspect, the parent bacterial cell is a Streptomyces coelicolor cell. In another aspect, the parent bacterial cell is a Streptomyces griseus cell. In another aspect, the parent bacterial cell is a Streptomyces lividans cell.

In the methods of the present invention, the parent bacterial cell may be any E. coli cell.

In another aspect of the present invention, the parent bacterial cell may additionally contain modifications, e.g., deletions or disruptions, of other genes that may be detrimental to the production, recovery or application of a heterologous polypeptide of interest. In a preferred aspect, the parent bacterial cell is a protease-deficient cell.

In a preferred aspect, the parent Bacillus cell comprises a disruption or deletion of aprE and nprE. In another preferred aspect, the parent Bacillus cell does not produce spores. In another more preferred aspect, a parent Bacillus cell comprises a disruption or deletion of spoIIAC. In another preferred aspect, the parent Bacillus cell comprises a disruption or deletion of one of the genes involved in the biosynthesis of surfactin, e.g., srfA, srfB, srfC, and srfD. See, for example, U.S. Pat. No. 5,958,728. Other genes, e.g., the amyE gene, which may be detrimental to the production, recovery or application of a polypeptide of interest may also be disrupted or deleted.

### Construction of Thiol-Disulfide Oxidoreductase-Deficient Bacterial Mutant Cells

The thiol-disulfide oxidoreductase-deficient bacterial mutant cell may be constructed by reducing or eliminating expression of a thiol-disulfide oxidoreductase gene in a parent bacterial cell using methods well known in the art, for example, insertions, disruptions, replacements, or deletions. The portion of the gene to be modified or inactivated may be, for example, the coding region or a regulatory element required for expression of the coding region. An example of such a regulatory or control sequence may be a promoter sequence or a functional part thereof, i.e., a part which is sufficient for affecting expression of the nucleic acid sequence. Other control sequences for possible modification include, but are not limited to, a leader, propeptide sequence, signal sequence, transcriptional terminator, and transcriptional activator.

The bacterial mutant cells may be constructed by gene deletion techniques to eliminate or reduce expression of a gene encoding a thiol-disulfide oxidoreductase. Gene deletion techniques enable the partial or complete removal of the thiol-disulfide oxidoreductase gene thereby eliminating their expression. In such methods, the deletion of the gene may be accomplished by homologous recombination using a plasmid that has been constructed to contiguously contain the 5′ and 3′ regions flanking the gene. The contiguous 5′ and 3′ regions may be introduced into a bacterial cell, for example, on a temperature-sensitive plasmid, such as pE194, at a temperature that allows the plasmid to become established in the cell. The cell is then shifted to a non-permissive temperature to select for cells that have the plasmid integrated into the chromosome at one of the homologous flanking regions. Selection for integration of the plasmid is effected by selection for the selectable marker. After integration, a recombination event at the second homologous flanking region is stimulated by shifting the cells to the permissive temperature for several generations without selection. The cells are plated to obtain single colonies and the colonies are examined for loss of the selectable marker (see, for example, Perego, 1993, In A. L. Sonneshein, J. A. Hoch, and R. Losick, editors, Bacillus subtilis and Other Gram-Positive Bacteria, Chapter 42, American Society of Microbiology, Washington, D.C.).

The bacterial mutant cells may be constructed by deletion of a thiol-disulfide oxidoreductase gene by simply replacing the region of the chromosome comprising the gene to be deleted with a selectable marker. This can be accomplished by cloning into a plasmid the 5′ and 3′ regions that flank the gene to be deleted and inserting a selectable marker in between these two regions. Once such a plasmid is constructed, it is linearized by digesting with a restriction enzyme that cuts outside of these 5′ and 3′ flanking regions. The linear DNA is then used to transform the bacterial cell selecting for the presence of the selectable marker contained between the two regions of flanking DNA. The only way the selectable marker can be incorporated into the genome is by a double crossover event, thereby replacing the gene to be deleted with the selectable marker.

The bacterial mutant cells may also be constructed by introducing, substituting, or removing one or more (several) nucleotides in a gene encoding a thiol-disulfide oxidoreductase or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortie, 1985, Science 229: 4719; Lo et al., 1985, Proceedings of the National Academy of Sciences USA 81: 2285; Higuchi et al., 1988, Nucleic Acids Research 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The bacterial mutant cells may also be constructed by gene disruption techniques by inserting into a gene encoding a thiol-disulfide oxidoreductase an integrative plasmid containing a nucleic acid fragment homologous to the gene which will create a duplication of the region of homology and incorporate vector DNA between the duplicated regions. Such a gene disruption can eliminate gene expression if the inserted vector separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The bacterial mutant cells may also be constructed by the process of gene conversion (see, for example, Iglesias and Trautner, 1983, Molecular General Genetics 189: 73-76). For example, in the gene conversion method, a nucleic acid sequence corresponding to the gene is mutagenized in vitro to produce a defective nucleic acid sequence, which is then transformed into the parent bacterial cell, e.g., a Bacillus cell, to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (see, for example, Perego, 1993, supra). Alternatively, the defective nucleic acid sequence may contain an insertion, substitution, or deletion of one or more (several) nucleotides of the gene, as described below.

The bacterial mutant cells may also be constructed by established anti-sense techniques using a nucleotide sequence complementary to the nucleic acid sequence of the gene (Parish and Stoker, 1997, FEMS Microbiology Letters 154: 151-157). More specifically, expression of the gene by a bacterial cell, e.g., a Bacillus cell, may be reduced or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The bacterial mutant cells may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970) and transposition (see, for example, Youngman et al., 1983, Proc. Natl. Acad. Sci. USA 80: 2305-2309). Modification of the gene may be performed by subjecting the parent cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent bacterial cell to be mutagenized in the presence of the mutagenizing agent of choice under suitable conditions, and selecting for mutant cells exhibiting reduced or no expression of the gene.

In a preferred embodiment, the modification of a gene encoding a thiol-disulfide oxidoreductase in the bacterial mutant cell is unmarked with a selectable marker. Removal of the selectable marker gene may be obtained by culturing the mutant cell on a counter-selection medium. Where the selectable marker gene contains repeats flanking its 5′ and 3′ ends, the repeats will facilitate the looping out of the selectable marker gene by homologous recombination when the mutant cell is submitted to counter-selection. The selectable marker gene may also be removed by homologous recombination by introducing into the mutant cell a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

It will be understood that the methods of the present invention are not limited to a particular order for obtaining the bacterial mutant cells. Modification of the gene encoding a thiol-disulfide oxidoreductase may be introduced into a parent cell at any step in the construction of the mutant cell for the production of a heterologous polypeptide.

In one aspect, a bdbC gene or homolog thereof is modified. In another aspect, a bdbD gene or homolog thereof is modified. In another aspect, a bdbC gene or homolog thereof and a bdbD gene or homolog thereof are modified. In another aspect, one or more (several) bdbC genes and/or one or more (several) bdbD genes (or thiol-disulfide oxidoreductase genes) are modified. In another aspect, the bdbC gene comprises SEQ ID NO: 1, SEQ ID NO: 3, SEQ ID NO: 49, SEQ ID NO: 51, SEQ ID NO: 53, SEQ ID NO: 55, SEQ ID NO: 57, or SEQ ID NO: 59. In another aspect, the bdbD gene comprises SEQ ID NO: 3, SEQ ID NO: 61, SEQ ID NO: 63, SEQ ID NO: 65, or SEQ ID NO: 67.

### Thiol-Disulfide Oxidoreductases and Genes Thereof

In the methods of the present invention, the thiol-disulfide oxidoreductase can be any thiol-disulfide oxidoreductase that adversely affects production of a heterologous polypeptide by incorrectly catalyzing the formation of one or more (several) disulfide bonds between two or more (several) cysteines of the heterologous polypeptide resulting in the polypeptide having no or less biological activity.

In a first aspect, the thiol-disulfide oxidoreductases comprise an amino acid sequence having a degree of sequence identity to the polypeptide of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have thiol-disulfide oxidoreductase activity. In one aspect, the polypeptides differ by no more than ten amino acids, e.g., by nine amino acids, by eight amino acids, by seven amino acids, by six amino acids, by five amino acids, by four amino acids, by three amino acids, by two amino acids, and by one amino acid from the mature polypeptide of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68.

In one embodiment, the thiol-disulfide oxidoreductase preferably comprises or consists of the amino acid sequence of SEQ ID NO: 2 or an allelic variant thereof; or a fragment thereof having thiol-disulfide oxidoreductase activity. In one aspect, the thiol-disulfide oxidoreductase comprises or consists of the amino acid sequence of SEQ ID NO: 2. In another aspect, the thiol-disulfide oxidoreductase comprises or consists of the mature polypeptide of SEQ ID NO: 2.

In another embodiment, the thiol-disulfide oxidoreductase preferably comprises or consists of the amino acid sequence of SEQ ID NO: 4 or an allelic variant thereof; or a fragment thereof having thiol-disulfide oxidoreductase activity. In one aspect, the thiol-disulfide oxidoreductase comprises or consists of the amino acid sequence of SEQ ID NO: 4. In another aspect, the thiol-disulfide oxidoreductase comprises or consists of the mature polypeptide of SEQ ID NO: 4.

In one embodiment, the thiol-disulfide oxidoreductase preferably comprises or consists of the amino acid sequence of SEQ ID NO: 50 or an allelic variant thereof; or a fragment thereof having thiol-disulfide oxidoreductase activity. In one aspect, the thiol-disulfide oxidoreductase comprises or consists of the amino acid sequence of SEQ ID NO: 50. In another aspect, the thiol-disulfide oxidoreductase comprises or consists of the mature polypeptide of SEQ ID NO: 50.

In one embodiment, the thiol-disulfide oxidoreductase preferably comprises or consists of the amino acid sequence of SEQ ID NO: 52 or an allelic variant thereof; or a fragment thereof having thiol-disulfide oxidoreductase activity. In one aspect, the thiol-disulfide oxidoreductase comprises or consists of the amino acid sequence of SEQ ID NO: 52. In another aspect, the thiol-disulfide oxidoreductase comprises or consists of the mature polypeptide of SEQ ID NO: 52.

In one embodiment, the thiol-disulfide oxidoreductase preferably comprises or consists of the amino acid sequence of SEQ ID NO: 54 or an allelic variant thereof; or a fragment thereof having thiol-disulfide oxidoreductase activity. In one aspect, the thiol-disulfide oxidoreductase comprises or consists of the amino acid sequence of SEQ ID NO: 54. In another aspect, the thiol-disulfide oxidoreductase comprises or consists of the mature polypeptide of SEQ ID NO: 54.

In one embodiment, the thiol-disulfide oxidoreductase preferably comprises or consists of the amino acid sequence of SEQ ID NO: 56 or an allelic variant thereof; or a fragment thereof having thiol-disulfide oxidoreductase activity. In one aspect, the thiol-disulfide oxidoreductase comprises or consists of the amino acid sequence of SEQ ID NO: 56. In another aspect, the thiol-disulfide oxidoreductase comprises or consists of the mature polypeptide of SEQ ID NO: 56.

In one embodiment, the thiol-disulfide oxidoreductase preferably comprises or consists of the amino acid sequence of SEQ ID NO: 58 or an allelic variant thereof; or a fragment thereof having thiol-disulfide oxidoreductase activity. In one aspect, the thiol-disulfide oxidoreductase comprises or consists of the amino acid sequence of SEQ ID NO: 58. In another aspect, the thiol-disulfide oxidoreductase comprises or consists of the mature polypeptide of SEQ ID NO: 58.

In one embodiment, the thiol-disulfide oxidoreductase preferably comprises or consists of the amino acid sequence of SEQ ID NO: 60 or an allelic variant thereof; or a fragment thereof having thiol-disulfide oxidoreductase activity. In one aspect, the thiol-disulfide oxidoreductase comprises or consists of the amino acid sequence of SEQ ID NO: 60. In another aspect, the thiol-disulfide oxidoreductase comprises or consists of the mature polypeptide of SEQ ID NO: 60.

In one embodiment, the thiol-disulfide oxidoreductase preferably comprises or consists of the amino acid sequence of SEQ ID NO: 62 or an allelic variant thereof; or a fragment thereof having thiol-disulfide oxidoreductase activity. In one aspect, the thiol-disulfide oxidoreductase comprises or consists of the amino acid sequence of SEQ ID NO: 62. In another aspect, the thiol-disulfide oxidoreductase comprises or consists of the mature polypeptide of SEQ ID NO: 62.

In one embodiment, the thiol-disulfide oxidoreductase preferably comprises or consists of the amino acid sequence of SEQ ID NO: 64 or an allelic variant thereof; or a fragment thereof having thiol-disulfide oxidoreductase activity. In one aspect, the thiol-disulfide oxidoreductase comprises or consists of the amino acid sequence of SEQ ID NO: 64. In another aspect, the thiol-disulfide oxidoreductase comprises or consists of the mature polypeptide of SEQ ID NO: 64.

In one embodiment, the thiol-disulfide oxidoreductase preferably comprises or consists of the amino acid sequence of SEQ ID NO: 66 or an allelic variant thereof; or a fragment thereof having thiol-disulfide oxidoreductase activity. In one aspect, the thiol-disulfide oxidoreductase comprises or consists of the amino acid sequence of SEQ ID NO: 66. In another aspect, the thiol-disulfide oxidoreductase comprises or consists of the mature polypeptide of SEQ ID NO: 66.

In one embodiment, the thiol-disulfide oxidoreductase preferably comprises or consists of the amino acid sequence of SEQ ID NO: 68 or an allelic variant thereof; or a fragment thereof having thiol-disulfide oxidoreductase activity. In one aspect, the thiol-disulfide oxidoreductase comprises or consists of the amino acid sequence of SEQ ID NO: 68. In another aspect, the thiol-disulfide oxidoreductase comprises or consists of the mature polypeptide of SEQ ID NO: 68.

In a second aspect, the thiol-disulfide oxidoreductases are encoded by polynucleotides that hybridize under very low stringency conditions, low stringency conditions, medium stringency conditions, medium-high stringency conditions, high stringency conditions, or very high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof (J. Sambrook, E. F. Fritsch, and T. Maniatis, 1989, Molecular Cloning, A Laboratory Manual, 2d edition, Cold Spring Harbor, N.Y.).

The polynucleotide of SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or a subsequence thereof; as well as the amino acid sequence of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or a fragment thereof; may be used to design nucleic acid probes to identify and clone DNA encoding a thiol-disulfide oxidoreductase from strains of different genera or species according to methods well known in the art. In particular, such probes can be used for hybridization with the genomic DNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 700 nucleotides, even more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin).

A genomic DNA library prepared from such other strains may, therefore, be screened for DNA that hybridizes with the probes described above and encodes a thiol-disulfide oxidoreductase. Genomic DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67, or a subsequence thereof, the carrier material is preferably used in a Southern blot.

For purposes of the present invention, hybridization indicates that the polynucleotide hybridizes to a labeled nucleic acid probe corresponding to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; or a subsequence thereof; under very low to very high stringency conditions. Molecules to which the nucleic acid probe hybridizes under these conditions can be detected using, for example, X-ray film.

In one aspect, the nucleic acid probe is the mature polypeptide coding sequence of SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67. In another aspect, the nucleic acid probe is a polynucleotide that encodes the polypeptide of SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68, or a subsequence thereof. In another aspect, the nucleic acid probe is SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

In a third aspect, the thiol-disulfide oxidoreductases are encoded by polynucleotides comprising or consisting of nucleotide sequences having a degree of sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67, or the mature polypeptide coding sequence thereof, of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which encode thiol-disulfide oxidoreductases.

In one embodiment, the polynucleotide comprises or consists of SEQ ID NO: 1. In another embodiment, the polynucleotide comprises or consists of the mature polypeptide coding sequence of SEQ ID NO: 1. The present invention also encompasses polynucleotides that encode polypeptides comprising or consisting of the amino acid sequence of SEQ ID NO: 2 or the mature polypeptide thereof, which differ from SEQ ID NO: 1 or the mature polypeptide coding sequence thereof by virtue of the degeneracy of the genetic code. The present invention also relates to subsequences of SEQ ID NO: 1 that encode fragments of SEQ ID NO: 2 having thiol-disulfide oxidoreductase activity.

In another embodiment, the polynucleotide comprises or consists of SEQ ID NO: 3. In another embodiment, the polynucleotide comprises or consists of the mature polypeptide coding sequence of SEQ ID NO: 3. The present invention also encompasses polynucleotides that encode polypeptides comprising or consisting of the amino acid sequence of SEQ ID NO: 4 or the mature polypeptide thereof, which differ from SEQ ID NO: 3 or the mature polypeptide coding sequence thereof by virtue of the degeneracy of the genetic code. The present invention also relates to subsequences of SEQ ID NO: 3 that encode fragments of SEQ ID NO: 4 having thiol-disulfide oxidoreductase activity.

In another embodiment, the polynucleotide comprises or consists of SEQ ID NO: 49. In another embodiment, the polynucleotide comprises or consists of the mature polypeptide coding sequence of SEQ ID NO: 49. The present invention also encompasses polynucleotides that encode polypeptides comprising or consisting of the amino acid sequence of SEQ ID NO: 50 or the mature polypeptide thereof, which differ from SEQ ID NO: 49 or the mature polypeptide coding sequence thereof by virtue of the degeneracy of the genetic code. The present invention also relates to subsequences of SEQ ID NO: 49 that encode fragments of SEQ ID NO: 50 having thiol-disulfide oxidoreductase activity.

In another embodiment, the polynucleotide comprises or consists of SEQ ID NO: 51. In another embodiment, the polynucleotide comprises or consists of the mature polypeptide coding sequence of SEQ ID NO: 51. The present invention also encompasses polynucleotides that encode polypeptides comprising or consisting of the amino acid sequence of SEQ ID NO: 52 or the mature polypeptide thereof, which differ from SEQ ID NO: 51 or the mature polypeptide coding sequence thereof by virtue of the degeneracy of the genetic code. The present invention also relates to subsequences of SEQ ID NO: 51 that encode fragments of SEQ ID NO: 52 having thiol-disulfide oxidoreductase activity.

In another embodiment, the polynucleotide comprises or consists of SEQ ID NO: 53. In another embodiment, the polynucleotide comprises or consists of the mature polypeptide coding sequence of SEQ ID NO: 53. The present invention also encompasses polynucleotides that encode polypeptides comprising or consisting of the amino acid sequence of SEQ ID NO: 54 or the mature polypeptide thereof, which differ from SEQ ID NO: 53 or the mature polypeptide coding sequence thereof by virtue of the degeneracy of the genetic code. The present invention also relates to subsequences of SEQ ID NO: 53 that encode fragments of SEQ ID NO: 54 having thiol-disulfide oxidoreductase activity.

In another embodiment, the polynucleotide comprises or consists of SEQ ID NO: 55. In another embodiment, the polynucleotide comprises or consists of the mature polypeptide coding sequence of SEQ ID NO: 55. The present invention also encompasses polynucleotides that encode polypeptides comprising or consisting of the amino acid sequence of SEQ ID NO: 56 or the mature polypeptide thereof, which differ from SEQ ID NO: 55 or the mature polypeptide coding sequence thereof by virtue of the degeneracy of the genetic code. The present invention also relates to subsequences of SEQ ID NO: 55 that encode fragments of SEQ ID NO: 56 having thiol-disulfide oxidoreductase activity.

In another embodiment, the polynucleotide comprises or consists of SEQ ID NO: 57. In another embodiment, the polynucleotide comprises or consists of the mature polypeptide coding sequence of SEQ ID NO: 57. The present invention also encompasses polynucleotides that encode polypeptides comprising or consisting of the amino acid sequence of SEQ ID NO: 58 or the mature polypeptide thereof, which differ from SEQ ID NO: 57 or the mature polypeptide coding sequence thereof by virtue of the degeneracy of the genetic code. The present invention also relates to subsequences of SEQ ID NO: 57 that encode fragments of SEQ ID NO: 58 having thiol-disulfide oxidoreductase activity.

In another embodiment, the polynucleotide comprises or consists of SEQ ID NO: 59. In another embodiment, the polynucleotide comprises or consists of the mature polypeptide coding sequence of SEQ ID NO: 59. The present invention also encompasses polynucleotides that encode polypeptides comprising or consisting of the amino acid sequence of SEQ ID NO: 60 or the mature polypeptide thereof, which differ from SEQ ID NO: 59 or the mature polypeptide coding sequence thereof by virtue of the degeneracy of the genetic code. The present invention also relates to subsequences of SEQ ID NO: 59 that encode fragments of SEQ ID NO: 60 having thiol-disulfide oxidoreductase activity.

In another embodiment, the polynucleotide comprises or consists of SEQ ID NO: 61. In another embodiment, the polynucleotide comprises or consists of the mature polypeptide coding sequence of SEQ ID NO: 61. The present invention also encompasses polynucleotides that encode polypeptides comprising or consisting of the amino acid sequence of SEQ ID NO: 62 or the mature polypeptide thereof, which differ from SEQ ID NO: 61 or the mature polypeptide coding sequence thereof by virtue of the degeneracy of the genetic code. The present invention also relates to subsequences of SEQ ID NO: 61 that encode fragments of SEQ ID NO: 62 having thiol-disulfide oxidoreductase activity.

In another embodiment, the polynucleotide comprises or consists of SEQ ID NO: 63. In another embodiment, the polynucleotide comprises or consists of the mature polypeptide coding sequence of SEQ ID NO: 63. The present invention also encompasses polynucleotides that encode polypeptides comprising or consisting of the amino acid sequence of SEQ ID NO: 64 or the mature polypeptide thereof, which differ from SEQ ID NO: 63 or the mature polypeptide coding sequence thereof by virtue of the degeneracy of the genetic code. The present invention also relates to subsequences of SEQ ID NO: 63 that encode fragments of SEQ ID NO: 64 having thiol-disulfide oxidoreductase activity.

In another embodiment, the polynucleotide comprises or consists of SEQ ID NO: 65. In another embodiment, the polynucleotide comprises or consists of the mature polypeptide coding sequence of SEQ ID NO: 65. The present invention also encompasses polynucleotides that encode polypeptides comprising or consisting of the amino acid sequence of SEQ ID NO: 66 or the mature polypeptide thereof, which differ from SEQ ID NO: 65 or the mature polypeptide coding sequence thereof by virtue of the degeneracy of the genetic code. The present invention also relates to subsequences of SEQ ID NO: 65 that encode fragments of SEQ ID NO: 66 having thiol-disulfide oxidoreductase activity.

In another embodiment, the polynucleotide comprises or consists of SEQ ID NO: 67. In another embodiment, the polynucleotide comprises or consists of the mature polypeptide coding sequence of SEQ ID NO: 67. The present invention also encompasses polynucleotides that encode polypeptides comprising or consisting of the amino acid sequence of SEQ ID NO: 68 or the mature polypeptide thereof, which differ from SEQ ID NO: 67 or the mature polypeptide coding sequence thereof by virtue of the degeneracy of the genetic code. The present invention also relates to subsequences of SEQ ID NO: 67 that encode fragments of SEQ ID NO: 68 having thiol-disulfide oxidoreductase activity.

A polynucleotide homologous to the polynucleotides encoding thiol-disulfide oxidoreductases described herein may be used from other microbial sources which produce a thiol-disulfide oxidoreductase to modify the corresponding gene in a bacterial cell of choice.

The techniques used to isolate or clone a polynucleotide encoding a thiol-disulfide oxidoreductase are known in the art and include isolation from genomic DNA. The cloning of the polynucleotide from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT), and nucleotide sequence-based amplification (NASBA) may be used. The polynucleotide may be cloned from a bacterial strain, e.g., Bacillus, and thus, for example, may be an allelic or species variant of the polypeptide encoding region of the nucleotide sequence.

In one embodiment, the polynucleotide encoding a thiol-disulfide oxidoreductase preferably comprises or consists of a nucleotide sequence having a degree of sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67, or the mature polypeptide coding sequence thereof, of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which encode a thiol-disulfide oxidoreductase.

In another embodiment, the polynucleotide encoding a thiol-disulfide oxidoreductase preferably hybridizes under very low stringency conditions, low stringency conditions, medium stringency conditions, medium-high stringency conditions, high stringency conditions, or very high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; or allelic variants and subsequences thereof (Sambrook et al., 1989, supra), as defined herein.

In one aspect, the thiol-disulfide oxidoreductase gene is a bdbC gene encoding the thiol-disulfide oxidoreductase of SEQ ID NO: 2. In another aspect, the bdbC gene comprises a polynucleotide comprising or consisting of SEQ ID NO: 1.

In one aspect, the thiol-disulfide oxidoreductase gene is a bdbC gene encoding the thiol-disulfide oxidoreductase of SEQ ID NO: 50. In another aspect, the bdbC gene comprises a polynucleotide comprising or consisting of SEQ ID NO: 49.

In one aspect, the thiol-disulfide oxidoreductase gene is a bdbC gene encoding the thiol-disulfide oxidoreductase of SEQ ID NO: 52. In another aspect, the bdbC gene comprises a polynucleotide comprising or consisting of SEQ ID NO: 51.

In one aspect, the thiol-disulfide oxidoreductase gene is a bdbC gene encoding the thiol-disulfide oxidoreductase of SEQ ID NO: 54. In another aspect, the bdbC gene comprises a polynucleotide comprising or consisting of SEQ ID NO: 53.

In one aspect, the thiol-disulfide oxidoreductase gene is a bdbC gene encoding the thiol-disulfide oxidoreductase of SEQ ID NO: 56. In another aspect, the bdbC gene comprises a polynucleotide comprising or consisting of SEQ ID NO: 55.

In one aspect, the thiol-disulfide oxidoreductase gene is a bdbC gene encoding the thiol-disulfide oxidoreductase of SEQ ID NO: 58. In another aspect, the bdbC gene comprises a polynucleotide comprising or consisting of SEQ ID NO: 57.

In one aspect, the thiol-disulfide oxidoreductase gene is a bdbC gene encoding the thiol-disulfide oxidoreductase of SEQ ID NO: 60. In another aspect, the bdbC gene comprises a polynucleotide comprising or consisting of SEQ ID NO: 59.

In another aspect, the thiol-disulfide oxidoreductase gene is a bdbD gene encoding the thiol-disulfide oxidoreductase of SEQ ID NO: 4. In another aspect, the bdbD gene comprises a polynucleotide comprising or consisting of SEQ ID NO: 3.

In another aspect, the thiol-disulfide oxidoreductase gene is a bdbD gene encoding the thiol-disulfide oxidoreductase of SEQ ID NO: 62. In another aspect, the bdbD gene comprises a polynucleotide comprising or consisting of SEQ ID NO: 61.

In another aspect, the thiol-disulfide oxidoreductase gene is a bdbD gene encoding the thiol-disulfide oxidoreductase of SEQ ID NO: 64. In another aspect, the bdbD gene comprises a polynucleotide comprising or consisting of SEQ ID NO: 63.

In another aspect, the thiol-disulfide oxidoreductase gene is a bdbD gene encoding the thiol-disulfide oxidoreductase of SEQ ID NO: 66. In another aspect, the bdbD gene comprises a polynucleotide comprising or consisting of SEQ ID NO: 65.

In another aspect, the thiol-disulfide oxidoreductase gene is a bdbD gene encoding the thiol-disulfide oxidoreductase of SEQ ID NO: 68. In another aspect, the bdbD gene comprises a polynucleotide comprising or consisting of SEQ ID NO: 67.

### Polypeptides

The heterologous polypeptide can be any polypeptide having a biological activity of interest. The term “heterologous polypeptide” is defined herein as a polypeptide that is not native to the host cell; a native polypeptide in which structural modifications, e.g., deletions, substitutions, and/or insertions, have been made to alter the native polypeptide; or a native polypeptide whose expression is quantitatively altered as a result of manipulation of the DNA encoding the polypeptide by recombinant DNA techniques, e.g., a stronger promoter, multiple copies of the DNA, etc. The polypeptide may be a naturally occurring allelic and engineered variations of the below-mentioned polypeptides. The term “polypeptide” is not meant herein to refer to a specific length of the encoded product and, therefore, encompasses peptides, oligopeptides, and proteins.

In one aspect, the polypeptide is an antibody, an antigen, an antimicrobial peptide, an enzyme, a growth factor, a hormone, an immunodilator, a neurotransmitter, a receptor, a reporter protein, a structural protein, or a transcription factor.

In another aspect, the polypeptide is an oxidoreductase, a transferase, a hydrolase, a lyase, an isomerase, or a ligase.

In another aspect, the polypeptide is an alpha-glucosidase, aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

In another aspect, the polypeptide is an albumin, a collagen, a tropoelastin, an elastin, or a gelatin.

In another aspect, the polypeptide is a hybrid polypeptide comprising portions of two or more (several) polypeptide, for example, in which a portion of one polypeptide is fused at the N-terminus or the C-terminus of a portion of another polypeptide. One or more (several) of the polypeptides may be heterologous to the bacterial cell.

In another aspect, the polypeptide is a fused polypeptide or cleavable fusion polypeptide in which a polypeptide is fused at the N-terminus or the C-terminus of another polypeptide. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Nucleic Acid Constructs

A polynucleotide encoding a heterologous polypeptide of interest can be manipulated in a variety of ways to provide for expression of the polynucleotide in a bacterial mutant cell of the present invention. Manipulation of the polynucleotide's nucleotide sequence prior to its insertion into a nucleic acid construct or vector may be desirable or necessary depending on the nucleic acid construct or vector or bacterial mutant cell. The techniques for modifying nucleotide sequences utilizing cloning methods are well known in the art.

A nucleic acid construct comprising a polynucleotide encoding a heterologous polypeptide of interest may be operably linked to one or more (several) control sequences capable of directing the expression of the coding sequence in the bacterial mutant cell under conditions compatible with the control sequences.

Each control sequence may be native or foreign to the polynucleotide encoding a polypeptide of interest. Such control sequences include, but are not limited to, a leader, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide.

The control sequence may be an appropriate promoter sequence, a nucleotide sequence that is recognized by the bacterial mutant cell for expression of the polynucleotide. The promoter sequence contains transcription control sequences that mediate the expression of the polypeptide of interest. The promoter may be any nucleotide sequence that shows transcriptional activity in the bacterial mutant cell and may be obtained from genes directing synthesis of extracellular or intracellular polypeptides having biological activity either homologous or heterologous to the bacterial mutant cell.

Examples of suitable promoters for directing the transcription of the nucleic acid constructs in a bacterial cell are the promoters obtained from the E. coli lac operon, the Streptomyces coelicolor agarase gene (dagA), the Bacillus subtilis levansucrase gene (sacB), the Bacillus licheniformis alpha-amylase gene (amyL), the Bacillus stearothermophilus maltogenic amylase gene (amyM), the Bacillus amyloliquefaciens alpha-amylase gene (amyQ), the Bacillus licheniformis penicillinase gene (penP), the Bacillus subtilis xylA and xylB genes, and the prokaryotic beta-lactamase gene (Villa-Komaroff et al., 1978, Proceedings of the National Academy of Sciences USA 75:3727-3731), as well as the tac promoter (DeBoer et al., 1983, Proceedings of the National Academy of Sciences USA 80:21-25). Further promoters are described in “Useful proteins from recombinant bacteria” in Scientific American, 1980, 242:74-94; and in J. Sambrook, E. F. Fritsch, and T. Maniatis, 1989, Molecular Cloning, A Laboratory Manual, 2d edition, Cold Spring Harbor, N.Y.).

The control sequence may also be a suitable transcription terminator sequence, a sequence recognized by a bacterial cell to terminate transcription. The terminator sequence is operably linked to the 3′ terminus of the polynucleotide sequence encoding a polypeptide of interest. Any terminator that is functional in the bacterial mutant cell may be used in the present invention.

The control sequence may also be a suitable leader sequence, a nontranslated region of a mRNA that is important for translation by the bacterial cell. The leader sequence is operably linked to the 5′ terminus of the polynucleotide encoding the polypeptide having biological activity. Any leader sequence that is functional in the bacterial mutant cell may be used in the present invention.

The control sequence may also be a signal peptide coding region, which codes for an amino acid sequence linked to the amino terminus of a polypeptide that can direct the expressed polypeptide into the cell's secretory pathway. The signal peptide coding region may be native to the polypeptide or may be obtained from foreign sources. The 5′ end of the coding sequence of the polynucleotide may inherently contain a signal peptide coding region naturally linked in translation reading frame with the segment of the coding region that encodes the secreted polypeptide. Alternatively, the 5′ end of the coding sequence may contain a signal peptide coding region that is foreign to that portion of the coding sequence and encodes the secreted polypeptide. The foreign signal peptide coding region may be required where the coding sequence does not normally contain a signal peptide coding region. Alternatively, the foreign signal peptide coding region may simply replace the natural signal peptide coding region in order to obtain enhanced secretion of the polypeptide relative to the natural signal peptide coding region normally associated with the coding sequence. The signal peptide coding region may be obtained from an amylase or a protease gene from a Bacillus species. However, any signal peptide coding region capable of directing the expressed polypeptide into the secretory pathway of the bacterial mutant cell may be used in the present invention.

An effective signal peptide coding region for bacterial cells, e.g., Bacillus cells, is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis prsA gene. Further signal peptides are described by Simonen and Palva, 1993, Microbiological Reviews 57:109-137.

The control sequence may also be a mRNA stabilizing sequence. The term “mRNA stabilizing sequence” is defined herein as a sequence located downstream of a promoter region and upstream of a coding sequence of a polynucleotide to which the promoter region is operably linked such that all mRNAs synthesized from the promoter region may be processed to generate mRNA transcripts with a stabilizer sequence at the 5′ end of the transcripts. The presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of bacterial 16S ribosomal RNA. In one aspect, the mRNA processing/stabilizing sequence generates essentially single-size transcripts with a stabilizing sequence at the 5′ end of the transcripts. The mRNA processing/stabilizing sequence is preferably one that is complementary to the 3′ extremity of a bacterial 16S ribosomal RNA. See, U.S. Pat. Nos. 6,255,076 and 5,955,310.

An effective mRNA processing/stabilizing sequence for bacterial cells is the Bacillus thuringiensis cryIIIA mRNA processing/stabilizing sequence disclosed in WO 94/25612, or portions thereof, which retain the mRNA processing/stabilizing function, or the Bacillus subtilis SP82 mRNA processing/stabilizing sequence disclosed in Hue et al., 1995, Journal of Bacteriology 177: 3465-3471, or portions thereof, which retain the mRNA processing/stabilizing function.

The nucleic acid construct can then be introduced into a bacterial cell using methods known in the art or those methods described herein for expressing the polypeptide of interest.

### Recombinant Expression Vectors

In the methods of the present invention, a recombinant expression vector comprising a polynucleotide encoding a heterologous polypeptide of interest, a promoter, and transcriptional and translational stop signals may be used for the recombinant production of the polypeptide. The various nucleic acid and control sequences described above may be joined together to produce a recombinant expression vector that may include one or more (several) convenient restriction sites to allow for insertion or substitution of the polynucleotide encoding a polypeptide of interest at such sites. Alternatively, the polynucleotide may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression, and possibly secretion.

The recombinant expression vector may be any vector that can be conveniently subjected to recombinant DNA procedures and can bring about the expression of the polynucleotide. The choice of the vector will typically depend on the compatibility of the vector with the bacterial mutant cell into which the vector is to be introduced. The vectors may be linear or closed circular plasmids. The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the bacterial mutant cell, is integrated into the genome and replicated together with the chromosome(s) into which it has been integrated. The vector system may be a single vector or plasmid or two or more vectors or plasmids that together contain the total DNA to be introduced into the genome of the bacterial cell, or a transposon.

The vectors preferably contain an element(s) that permits integration of the vector into the bacterial cell genome or autonomous replication of the vector in the bacterial cell independent of the genome.

For integration into the bacterial cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of sequence identity to the corresponding target sequence to enhance the probability of homologous recombination. The integrational elements may be any sequence that is homologous with the target sequence in the genome of the bacterial cell. Furthermore, the integrational elements may be non-encoding or encoding nucleotide sequences.

For autonomous replication, the vector may further comprise an origin of replication enabling the vector to replicate autonomously in the bacterial cell. The origin of replication may be any plasmid replicator mediating autonomous replication that functions in the bacterial cell. The term “origin of replication” or “plasmid replicator” is defined herein as a nucleotide sequence that enables a plasmid or vector to replicate in vivo. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation to make its function temperature-sensitive in the bacterial cell (see, e.g., Ehrlich, 1978, Proceedings of the National Academy of Sciences USA 75:1433-1436).

More than one copy of a polynucleotide encoding a polypeptide of interest may be introduced into the bacterial cell to amplify expression of the polynucleotide. Stable amplification of the polynucleotide can be obtained by integrating at least one additional copy of the sequence into the bacterial cell genome using methods well known in the art and selecting for transformants. A convenient method for achieving amplification is described in WO 94/14968.

The vectors preferably contain one or more (several) selectable markers that permit easy selection of transformed cells. A selectable marker is a gene the product of which provides for biocide resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO 91/09129, where the selectable marker is on a separate vector.

The procedures used to ligate the elements described above to construct the recombinant expression vectors are well known to one skilled in the art (see, e.g., Sambrook et al., 1989, supra).

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-207, by electroporation (see, e.g., Buckley et al., 1999, Appl. Environ. Microbiol. 65: 3800-3804) or by conjugation (see, e.g., Clewell, 1981, Microbiol. Rev. 45: 409-436).

The present invention is further described by the following examples which should not be construed as limiting the scope of the invention.

### EXAMPLES

Bacillus subtilis strains were made competent using the method described by Anagnostopoulos and Spizizen, 1961, Journal of Bacteriology 81: 741-746.

DNA sequencing was conducted with an ABI 3700 Sequencing (Applied Biosystems, Inc., Foster City, Calif., USA).

### Media

2×YT agar plates were composed of 16 g of tryptone, 10 g of yeast extract, 5 g of sodium chloride, 15 g of Bactoagar, and deionized water to 1 liter.

LB medium was composed of 10 g of tryptone, 5 g of yeast extract, 10 g of sodium chloride, and deionized water to 1 liter (pH 7.4).

### Example 1

### Construction of Bacillus subtilis Strain SMO25

Bacillus subtilis strain SMO25 was constructed as described below to delete an intracellular serine protease (ispA) gene in Bacillus subtilis strain A164Δ10 (Bindel-Connelly et al., 2004, J. Bacteriol. 186: 4159-4167).

A deletion plasmid, pNNB194-ispAΔ, was constructed by splicing by overlap extension (SOE) (Horton et al., 1989, Gene 77: 61-8). Flanking DNA sequences 5′ and 3′ of the ispA gene were obtained by PCR amplification from chromosomal DNA derived from Bacillus subtilis strain 164Δ5 (U.S. Pat. No. 5,891,701) using primer pairs 994525/994526 and 994527/994528, respectively, shown below. Chromosomal DNA was obtained according to the procedure of Pitcher et al., 1989, Lett. Appl. Microbiol. 8: 151-156.

Primer 994525:
(SEQ ID NO: 5)
5′-GGATCCATTATGTAGGGCGTAAAGC-3′
Primer 994526:
(SEQ ID NO: 6)
5′-TTAGCAAGCTTAATCACTTTAATGCCCTCAG-3′
Primer 994527:
(SEQ ID NO: 7)
5′-TGATTAAGCTTGCTAATCCGCAGGACACTTC-3′
Primer 994528:
(SEQ ID NO: 8)
5′-GGTACCAACACTGCCTCTCTCATCTC-3′

PCR amplifications were conducted in 50 μl reactions composed of 10 ng of Bacillus subtilis strain 164Δ5 chromosomal DNA, 0.4 μM of each primer, 200 μM each of dATP, dCTP, dGTP, and dTTP, 1×PCR Buffer II (Applied Biosystems, Inc., Foster City, Calif., USA) with 2.5 mM MgCl₂, and 2.5 units of AmpliTaq GOLD® DNA Polymerase (Applied Biosystems, Inc., Foster City, Calif., USA). The reactions were performed in a ROBOCYCLER® 40 Temperature Cycler (Stratagene, Corp., La Jolla, Calif., USA) programmed for 1 cycle at 95° C. for 10 minutes; 25 cycles each at 95° C. for 1 minute, 50° C. for 1 minute, and 72° C. for 1 minute; and 1 cycle at 72° C. for 7 minutes.

The PCR products were resolved by 0.8% agarose gel electrophoresis using 0.5×TBE buffer (50 mM Tris base-50 mM boric acid-1 mM disodium EDTA). A band of approximately 400 bp obtained using the primer pair 994525/994526 for the 5′ flanking DNA sequence of the ispA gene was excised from the gel and extracted using a QIAQUICK® Gel Extraction Kit (QIAGEN Inc., Valencia, Calif., USA). A band of approximately 400 bp obtained using the primer pair 994527/994528 for the 3′ flanking DNA sequence of the ispA gene was excised from the gel and extracted using a QIAQUICK® Gel Extraction Kit.

The final SOE fragment was amplified using the same procedure above with the 400 bp fragments as templates and primers 994525 and 994528, shown above, to produce an ispA deletion fragment. The PCR product of approximately 800 bp was resolved by 0.8% agarose gel electrophoresis using 0.5×TBE buffer.

The final 800 bp SOE fragment was cloned into pCR®2.1 (Invitrogen, Inc., Carlsbad, Calif., USA) using a TA-TOPO® Cloning Kit (Invitrogen, Inc., Carlsbad, Calif., USA) and transformed into ONE SHOT® TOP10 Chemically Competent E. coli cells (Invitrogen, Inc., Carlsbad, Calif., USA) according to the manufacturer's instructions. Transformants were selected on 2×YT agar plates supplemented with 100 μg of ampicillin per ml and incubated at 37° C. for 16 hours. The DNA sequence of the cloned fragment was verified by DNA sequencing with M13 forward and reverse primers (Invitrogen, Inc., Carlsbad, Calif., USA). The plasmid was designated pCR®2.1-ispAΔ.

Plasmid pCR2.1-ispAΔ was digested with Bam HI and Asp718 and subjected to 0.8% agarose gel electrophoresis using 0.5×TBE buffer to isolate the ispA deletion fragment. A 800 bp fragment corresponding to the ispA deletion fragment was excised from the gel and extracted using a QIAQUICK® Gel Extraction Kit.

The temperature sensitive plasmid pNNB194 (pSK⁺/pE194; U.S. Pat. No. 5,958,728) was digested with Bam HI and Asp718 and resolved by 0.8% agarose gel electrophoresis using 0.5×TBE buffer to isolate the vector fragment. A 6.6 kb vector fragment of pNNB194 was excised from the gel and extracted using a QIAQUICK® Gel Extraction Kit.

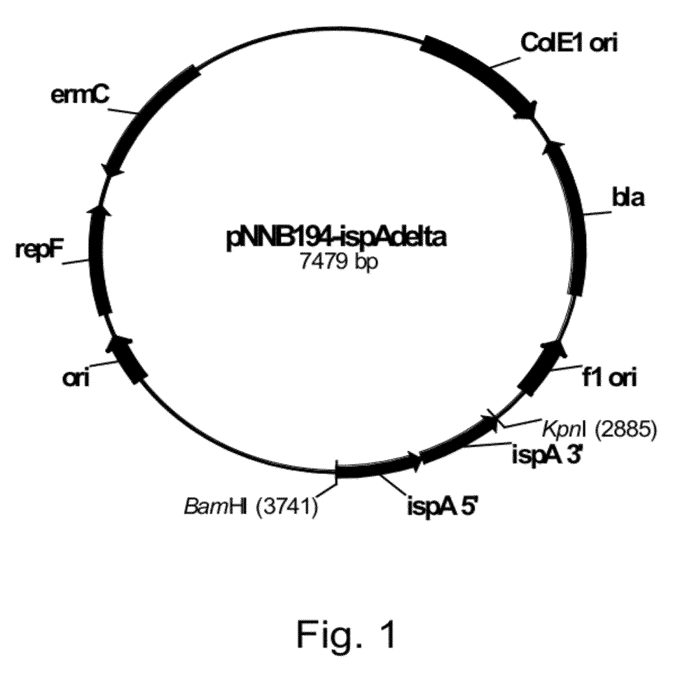

The ispA deletion fragment and the pNNB194 fragment were ligated together using a Rapid DNA Ligation Kit (Roche Applied Science, Indianapolis, Ind., USA) and the ligation mix was transformed into E. coli SURE® cells (Stratagene Corp., La Jolla, Calif., USA) selecting for ampicillin resistance according to the manufacturer's instructions. Plasmid DNA was isolated from eight transformants using a BIOROBOT® 9600 (QIAGEN Inc., Valencia, Calif., USA), digested with Bam HI and Asp718, and analyzed by agarose electrophoresis as described above to identify plasmids which harbored the ispAΔfragment. One transformant was identified and designated pNNB194-ispAΔ (FIG. 1).

Plasmid pNNB194-ispAΔ was introduced into Bacillus subtilis A164Δ10 (Bindel-Connelly et al., 2004, J. Bacteriol. 186: 4159-4167) and integrated at the ispA locus by selective growth at 45° C. on Tryptose blood agar base (TBAB) plates supplemented with 1 μg of erythromycin and 25 μg of lincomycin per ml. The integrated plasmid was then excised by non-selective growth on LB medium at 34° C. Chromosomal DNA was isolated from several erythromycin sensitive clones according to the method of Pitcher et al., 1989, supra, and analyzed by PCR using primers 994525 and 994528 according to the same method above to confirm the presence of the ispA deletion. One such clone was designated Bacillus subtilis SMO25.

### Example 2

### Construction of pRB219

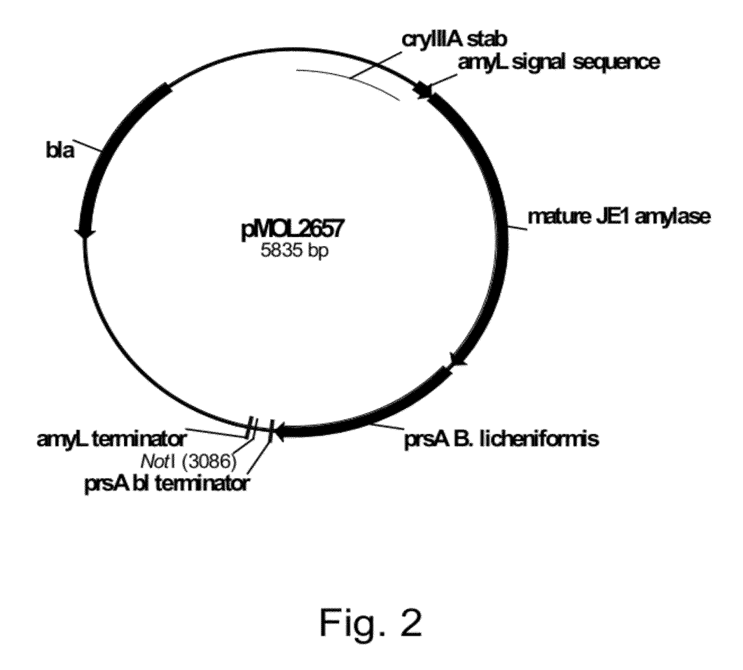

Plasmid pRB219 is based on pMOL2657 (FIG. 2; SEQ ID NO: 9), which is a pUC19-based plasmid harboring a transcriptional operon encoding JE1 alpha-amylase (WO 99/23211), a variant of the SP722 alpha-amylase, and the prsA chaperone from Bacillus licheniformis (Rey et al., 2004, Genome Biology 5:R77). The prsA gene encodes an essential membrane-bound lipoprotein that is assumed to assist post-translocational folding of exported proteins and stabilizes them in the compartment between the cytoplasmic membrane and cell wall. The transcriptional operon is preceded upstream by a mRNA stabilizing sequence from the Bacillus thuringiensis subsp. tenebrionis cryIIIA gene (WO 99/43835A), referred herein as “cryIIIA mRNA stabilizing sequence”. The JE1 alpha-amylase is fused to the signal peptide of the Bacillus licheniformis alpha-amylase (amyL) gene by a sequence inserted directly upstream from the maturation site (U.S. Patent Application 2009/0263881).

Plasmid rRB216.

Plasmid pMOL2657 was digested with Not I, and the ends were blunted by incubation for 20 minutes at 11° C. with T4 DNA polymerase (Roche Applied Science, Indianapolis, Ind., USA) and 25 μM each of dATP, dCTP, dGTP, and dTTP, followed by heat-inactivation of the T4 DNA polymerase by incubation for 10 minutes at 75° C. The digested plasmid was purified using a QIAQUICK® DNA Purification Kit (QIAGEN Inc., Valencia, Calif., USA) according to the manufacturer's instructions. The purified plasmid was treated with T4 DNA ligase and then transformed into E. coli XL-1 Blue competent cells (Invitrogen, Inc., Carlsbad, Calif., USA) according to manufacturer's instructions. Ampicillin-resistant transformants were selected on 2×YT plates supplemented with 100 μg of ampicillin per ml. Plasmid DNA was isolated from eight transformants using a BIOROBOT® 9600 and disruption of the Not I site was confirmed by the inability to digest the plasmid DNA with Not I. The plasmid DNA was also digested with Bgl II and analyzed by agarose electrophoresis as described above to confirm the identity of the plasmids. One plasmid with expected restriction fragments of approximately 3.2 kb and 2.7 kb was designated pRB216.

Plasmid rRB217.

Plasmid pRB217 was constructed by PCR amplification of the prsA gene from plasmid pRB216 using primers prsA-1F and prsA-2R, shown below. Primer prsA-1F incorporates Eco RI, Mlu I, and Hpa I restriction sites, while primer prsA-2R incorporates a Not I restriction site.

Primer prsA-1F:
(SEQ ID NO: 10)
5′-GAATTCACGCGTGTTAACTATGATTAGGAGTGTTTGCATT-3′
Primer prsA-2R:
(SEQ ID NO: 11)
5′-GCGGCCGCTATACTAGTTATCTCAACGAAATTTATAAGAC-3′

PCR amplifications were conducted in triplicate in 50 μl reactions composed of 1 ng of pRB216 DNA, 0.4 μM each of primers prsA-1F and prsA-2R, 200 μM each of dATP, dCTP, dGTP, and dTTP, 1×PCR Buffer II with 2.5 mM MgCl₂, and 2.5 units of AmpliTaq GOLD® DNA Polymerase. The reactions were performed in a ROBOCYCLER® 40 Temperature Cycler programmed for 1 cycle at 95° C. for 2 minutes; 30 cycles each at 95° C. for 1 minute, 55° C. for 1 minute, and 72° C. for 3 minute; and 1 cycle at 72° C. for 3 minutes.

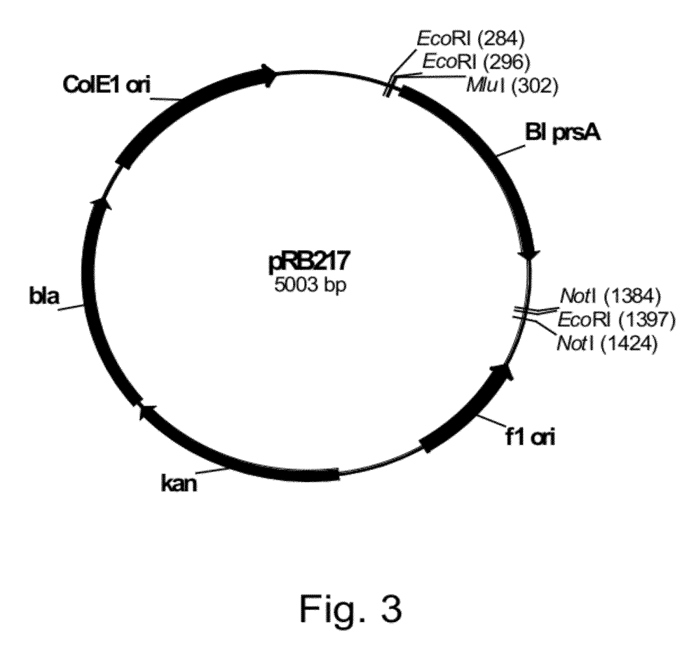

The PCR products were isolated by 0.8% agarose gel electrophoresis using 0.5×TBE buffer. A band of approximately 1.0 kb was excised from the gel and extracted using a QIAQUICK® Gel Extraction Kit. The resulting PCR product was cloned into pCR®2.1-TOPO® using a TOPO® TA Cloning Kit according to the manufacturer's instructions and transformed into ONE SHOT® TOP10 Chemically Competent E. coli cells according to the manufacturer's instructions. Transformants were selected on 2×YT agar plates supplemented with 100 μg of ampicillin per ml. Plasmid DNA was isolated from eight transformants using a BIOROBOT® 9600 and verified to contain the prsA fragment by Eco RI digestion followed by 0.8% agarose gel electrophoresis using 0.5×TBE buffer. One plasmid with expected restriction fragments of approximately 3.9 kb and 1.0 kb was identified and designated pRB217 (FIG. 3). The DNA sequence of the cloned fragment was verified by DNA sequencing using M13 forward and reverse primers.

Plasmid pRB219.

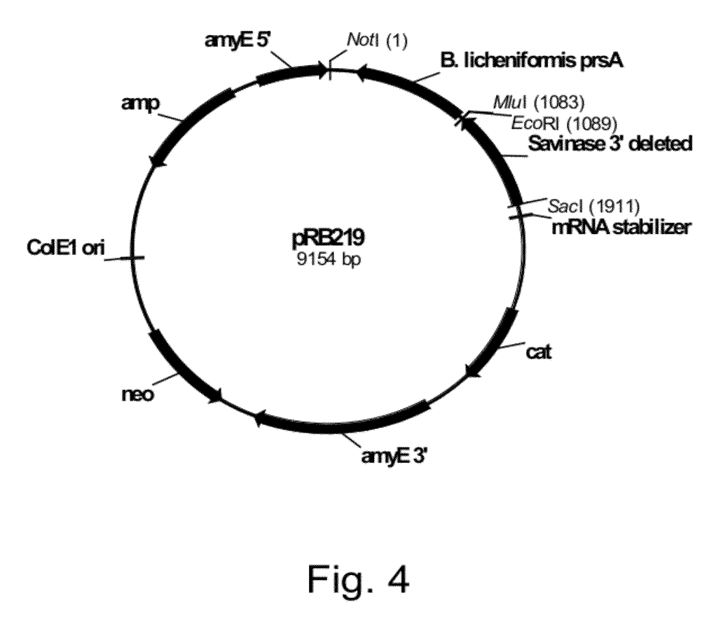

Plasmids pDG268MCSΔneo-cryIIIA stab/SAV (U.S. Pat. No. 5,955,310) and pRB217 were digested with Eco RI and Not I. The digested plasmids were subjected to 0.8% agarose gel electrophoresis using 0.5×TBE buffer. An 8.0 kb vector fragment from pDG268Δneo-cryIIIA stab/SAV and a 1.0 kb prsA insert fragment from pRB217 were excised from the gels and extracted using a QIAQUICK® Gel Extraction Kit. The vector fragment and prsA insert fragment were ligated together using a Rapid DNA Ligation Kit and the ligation mix was transformed into E. coli SURE® cells selecting for ampicillin resistance according to the manufacturer's instructions. Plasmid DNA was purified from several transformants using a BIOROBOT® 9600 and analyzed by Xho I digestion followed by 0.8% agarose gel electrophoresis using 0.5×TBE buffer. One plasmid with expected restriction fragments of approximately 5.5 kb and 3.5 kb was identified and designated pRB219 (FIG. 4).

### Example 3

### Construction of Bacillus subtilis EXP01955

A linear integration vector-system was used for the expression cloning of a synthetic version of the wild-type Dictyoglomus thermophilum Family 11 xylanase gene (SEQ ID NO: 12 [DNA sequence] and SEQ ID NO: 13 [deduced amino acid sequence]). The synthetic gene encodes a protein without a binding domain and without a signal peptide (SEQ ID NO: 14 [DNA sequence] and SEQ ID NO: 15 [deduced amino acid sequence]). The synthetic gene sequence was based on the public gene sequence UNIPROT: P77853. The synthetic gene was codon optimized for expression in Bacillus subtilis following recommendations by Gustafsson et al., 2004, Trends in Biotechnology 22: 346-353. The synthetic gene was generated by DNA2.0 (Menlo Park, Calif., USA) and delivered as a cloned fragment in their standard cloning vector (kanamycin resistant). The xylanase gene was cloned as a truncated gene without a binding domain and with the signal peptide from a Bacillus clausii serine protease gene (aprH, SAVINASE™, Novo Nordisk A/S, Bagsværd, Denmark) (included in the flanking region). The gene was designed to contain a C-terminal HQHQHQHQP tag to ease purification. The forward primer was designed so the gene was amplified from the signal peptide cleavage site and has 26 bases overhang (shown in italics in the table below). This overhang was complementary to part of one of the two linear vector fragments and was used when the PCR fragment and the vector fragments were assembled (described below). The reverse primer was designed to amplify the truncated version of the gene and contained an overhang consisting of 30 bp encoding the HQHQHQHQP-tag and a stop codon (the overhang is shown in italics in the table below). This overhang was complementary to part of one of the two linear vector fragments and was used when the PCR fragment and the vector fragments were assembled (described below).

The linear integration construct was a PCR fusion product made by fusion of each gene between two Bacillus subtils homologous chromosomal regions along with a strong promoter and a chloramphenicol resistance marker. The fusion was made by splicing by overlap extension (SOE) (Horton et al., 1989, supra). The SOE PCR method is also described in WO 2003/095658. Each gene was expressed under the control of a triple promoter system (described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including the mRNA stabilizing sequence. The gene coding for chloramphenicol acetyl-transferase was used as marker (described, for example, by Diderichsen et al., 1993, Plasmid 30: 312). The final gene construct was integrated by homologous recombination into the pectate lyase locus of the Bacillus chromosome.

The GH11 xylanase gene was amplified from plasmid 7587 by PCR using the primers shown in the Table 1 below. Plasmid 7587 contains the synthetic Dictyoglomus thermophilum GH11 xylanase gene (SEQ ID NO: 14) without a binding domain and without a signal peptide.

Three fragments were PCR amplified to make the construct: the gene fragment containing the truncated xylanase gene and the 26 bp and 30 bp flanking DNA sequences included in the primers as overhang, the upstream flanking fragment (including the signal peptide sequence from the Bacillus clausii aprH gene and amplified with primers 260558 and iMB1361Uni2) and the downstream flanking fragment (amplified with primers 260559 and HQHQHQHQP-f). The flanking fragments were amplified from genomic DNA of strain iMB1361 (described in Example 4 of WO 2003/095658). All primers used are listed in the Table 1 below.

The gene fragment was amplified using PHUSION™ DNA Polymerase (Finnzymes, Finland) according to the manufacturer's instructions. The two flanking DNA fragments were amplified using an EXPAND® High Fidelity PCR System (Roche-Applied-Science, Indianapolis, Ind., USA) according to the manufacturer's recommendations. The PCR conditions were as follows: 1 cycle at 94° C. for 2 minutes; 10 cycles each at 94° C. for 15 seconds, 50° C. for 45 seconds, and 68° C. for 4 minutes; 20 cycles each at 94° C. for 15 seconds, 50° C. for 45 seconds, and 68° C. for 4 minutes (+20 seconds extension per cycle); and 1 cycle at 68° C. for 10 minutes. The 3 PCR fragments were subjected to a subsequent Splicing by Overlap Extension (SOE) PCR reaction to assemble the 3 fragments into one linear vector construct. The SOE was performed by mixing the 3 fragments in equal molar ratios and a new PCR reaction was run under the following conditions: 1 cycle at 94° C. for 2 minutes; 10 cycles each at 94° C. for 15 seconds, 50° C. for 45 seconds, and 68° C. for 5 minutes; 10 cycles each at 94° C. for 15 seconds, 50° C. for 45 seconds, and 68° C. for 8 minutes; and 15 cycles each at 94° C. for 15 seconds, 50° C. for 45 seconds, and 68° C. for 8 minutes (in addition 20 seconds extra per cycle). After the 1^{st} cycle the two end primers 260558 and 260559 were added (20 pMol of each). Two μl of the PCR product were transformed into Bacillus subtilis PL4250 (AprE-, NprE-, SrfC-, SpoIIAC-, AmyE-, comS+). Transformants were selected on LB plates supplemented with 6 μg of chloramphenicol per ml. The truncated xylanase construct was integrated by homologous recombination into the genome of Bacillus subtilis PL4250. One transformant, EXP01955, was selected for further work. The xylanase coding region was sequenced in this transformant and found to contain one mutation leading to a change of the HQHQHQHQP-tag to a HQHQHQHQQ-tag, but no other mutations were observed.

TABLE 1
Primers

| | SPECIFIC PRIMER | SPECIFIC PRIMER |
|---|---|---|
| Amplification of | FORWARD | REVERSE |
| Truncated gene | Forward (SEQ ID NO: 16) | Reverse (SEQ ID NO: 17) |
| | 5′-CTTTTAGTTCATCGATCGC | 5′-CTAGGGTTGATGCTGGTG |
| | ATCGGCTGCTCAGACATCAA | TTGGTGCTGATGGCTGCCC |
| | TCACACTTA-3′ | TGAGAGAAAGTG-3′ |
| Upstream flanking | 260558: (SEQ ID NO: 18) | iMB1361Uni2 (SEQ ID NO: 19) |
| fragment | 5′-GAGTATCGCCAGTAAGG | 5′-AGCCGATGCGATCGATGA |
| | GGCG-3′ | ACTA-3′ |
| Downstream flanking | HQHQHQHQP-f (SEQ ID NO: | 260559: (SEQ ID NO: 21) |
| fragment | 20) | 5′-GCAGCCCTAAAATCGCAT |
| | 5′-CATCAGCACCAACACCAG | AAAGC-3′ |
| | CACCAGCCATAATCGCATGT | |
| | TCAATCCGCTCCATA-3′ | |

### Example 4

### Construction of Bacillus subtilis SMO59

Chromosomal DNA from Bacillus subtilis strain EXP01955 was used as a template to PCR clone the Bacillus clausii serine protease gene (aprH, SAVINASE™, Novo Nordisk A/S, Bagsværd, Denmark) signal sequence/mature D. thermophilum xylanase gene (CBM-deleted) into pCR®2.1-TOPO® using the following primers, which introduce a Sac I site at the 5′ end (just upstream of the aprH ribosome binding site) and a Mlu I site at the 3′ end (just after the translation stop codon which was introduced after the Ser codon at position 691-693, thereby avoiding the incorporation of the HQHQHQHQQ-tag). Chromosomal DNA was obtained according to the procedure of Pitcher et al., 1989, supra.

Primer 062405:
(SEQ ID NO: 22)
5′-GAGCTCTATAAAAATGAGGAGGGAACCGAATGAAGAAACC-3′
Primer 062406:
(SEQ ID NO: 23)
5′-ACGCGTTTAGCTGCCCTGAGAGAAAGTG-3′

The PCR amplifications were conducted in 50 μl reactions composed of 10 ng of B. subtilis EXP01955 chromosomal DNA, 0.4 μM of each primer, 200 μM each of dATP, dCTP, dGTP, and dTTP, 1×PCR Buffer II with 2.5 mM MgCl₂, and 2.5 units of AmpliTaq GOLD® DNA Polymerase. The reactions were performed in a ROBOCYCLER® 40 Temperature Cycler programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. A PCR product of approximately 740 bp of the truncated xylanase gene was resolved by 0.8% agarose gel electrophoresis using 0.5×TBE buffer, excised from the gel, and extracted using a QIAQUICK® Gel Extraction Kit.

The 740 bp fragment was cloned into pCR®2.1 using a TA-TOPO® Cloning Kit according to the manufacturer's instructions and transformed into ONE SHOT® TOP10 Chemically Competent E. coli cells according to the manufacturer's instructions. Transformants were selected on 2×YT agar plates supplemented with 100 μg of ampicillin per ml and incubated at 37° C. for 16 hours. The DNA sequence of the cloned fragment was verified by DNA sequencing with M13 forward and reverse primers. The plasmid was designated pCR2.1-Dt xyl.

DNA sequencing revealed that there was an extra G at position 19 of the sequence encoding the aprH signal sequence. A QUIKCHANGE® XL Site-Directed Mutagenesis Kit (Stratagene Corp., La Jolla, Calif., USA) was utilized to correct the error in plasmid pCR2.1-Dt xyl using the following primers to delete the extra G residue:

Primer 062535:
(SEQ ID NO: 24)
5′-CCGTTGGGGAAAATTGTCGC-3′
Primer 062536:
(SEQ ID NO: 25)
5′-GCGACAATTTTCCCCAACGG-3′

The kit was used according to the manufacturer's instructions and the change was successfully made resulting in plasmid pCR2.1-Dt xyl2. Plasmid pCR2.1-Dt xyl2 comprises the Bacillus clausii serine protease signal sequence linked to the mature D. thermophilum xylanase (CBM-deleted) without the HQHQHQHQQ-tag (SEQ ID NO: 26 and SEQ ID NO: 27)

Plasmids pCR2.1-Dt xyl2 and pRB219 were digested with Sac I and Mlu I. The digestions were each subjected to 0.8% agarose gel electrophoresis using 0.5×TBE buffer. A vector fragment of approximately 8.3 kb from pRB219 and a xylanase gene fragment of approximately 700 bp from pCR2.1-Dt xyl2 were excised from the gels and extracted using a QIAQUICK® Gel Extraction Kit.

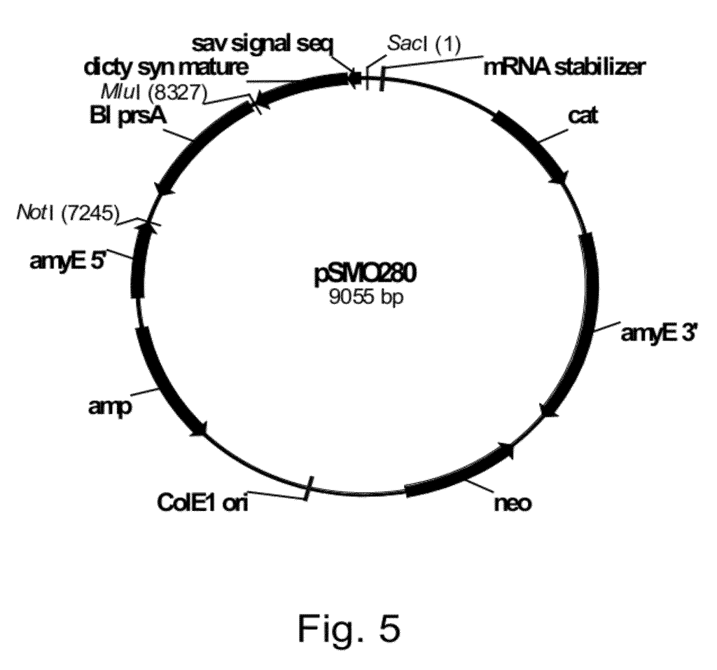

The two purified fragments were ligated together using a Rapid DNA Ligation Kit and the ligation mix was transformed into E. coli SURE® competent cells according to the manufacturer's instructions. Transformants were selected on 2×YT agar plates supplemented with 100 μg of ampicillin per ml at 37° C. Plasmids were purified from several transformants using a BIOROBOT® 9600 and analyzed by Sac I plus Mlu I digestion. The digestions were resolved by 0.8% agarose gel electrophoresis using 0.5×TBE buffer. A plasmid was identified by the presence of an approximately 700 bp Sac I-Mlu I fragment and designated pSMO280 (FIG. 5).

Plasmids pSMO280 and pMDT100 (WO 2008/140615 were digested with Sac I and Not I. The digestions were each resolved by 0.8% agarose gel electrophoresis using 0.5×TBE buffer. A vector fragment of approximately 8.0 kb from pMDT100 and a xylanase/prsA gene fragment of approximately 1.8 kb from pSMO280 were excised from the gels and extracted using a QIAQUICK® Gel Extraction Kit. The two purified fragments were ligated together using a Rapid DNA Ligation Kit.

Competent cells of Bacillus subtilis 168Δ4 were transformed with the ligation products according to the method of Young and Spizizen, 1961, Journal of Bacteriology 81: 823-829, or Dubnau and Davidoff-Abelson, 1971, Journal of Molecular Biology 56: 209-221. Bacillus subtilis 168Δ4 is derived from the Bacillus subtilis type strain 168 (BGSC 1A1, Bacillus Genetic Stock Center, Columbus, Ohio, USA) and has deletions in the spoIIAC, aprE, nprE, and amyE genes. The deletion of the four genes was performed essentially as described for Bacillus subtilis A164Δ5 (U.S. Pat. No. 5,891,701).

Bacillus subtilis transformants were selected at 37° C. after 16 hours of growth on TBAB plates supplemented with 5 μg of chloramphenicol per ml. To screen for integration of the plasmid by double cross-over at the amyE locus, Bacillus subtilis primary transformants were patched onto TBAB plates supplemented with 6 μg of neomycin per ml and onto TBAB plates supplemented with 5 μg of chloramphenicol per ml. Integration of the plasmid by double cross-over at the amyE locus does not incorporate the neomycin resistance gene and therefore renders the strain neomycin sensitive. A chloramphenicol resistant, neomycin sensitive transformant was identified, which harbored the Dictyoglomus thermophilum xylanase expression cassette in the amyE locus, and designated Bacillus subtilis SMO57.

Genomic DNA was isolated from Bacillus subtilis SMO57 (Pitcher et al., 1989, supra) and 0.1 μg was transformed into competent Bacillus subtilis SMO25. Transformants were selected on TBAB plates supplemented with 5 μg of chloramphenicol per ml at 37° C. A chloramphenicol resistant transformant was single colony purified and designated Bacillus subtilis SMO59.

### Example 5

### Construction of Bacillus subtilis MATA31

Plasmid pDN1981 (Jørgensen et al., 1990, Gene, 96: 37-41) was digested with Nde I. The ends were blunted by incubation for 20 minutes at 11° C. with T4 DNA polymerase and 25 μM each of dATP, dCTP, dGTP, and dTTP, followed by heat-inactivation of the T4 DNA polymerase by incubation for 10 minutes at 75° C., and then digested with Hind III. The digestion was resolved by 0.8% agarose gel electrophoresis using 0.5×TBE buffer. An amyL fragment of approximately 1.8 kb was excised from the gel and extracted using a QIAQUICK® Gel Extraction Kit. Plasmid pIC20R (Marsh et al., 1984, Gene, 32: 481-485) was digested with Hind III and Sma I. The digestion was resolved by 0.8% agarose gel electrophoresis using 0.5×TBE buffer. A vector fragment of approximately 2.7 kb was excised from the gel and extracted using a QIAQUICK® Gel Extraction.

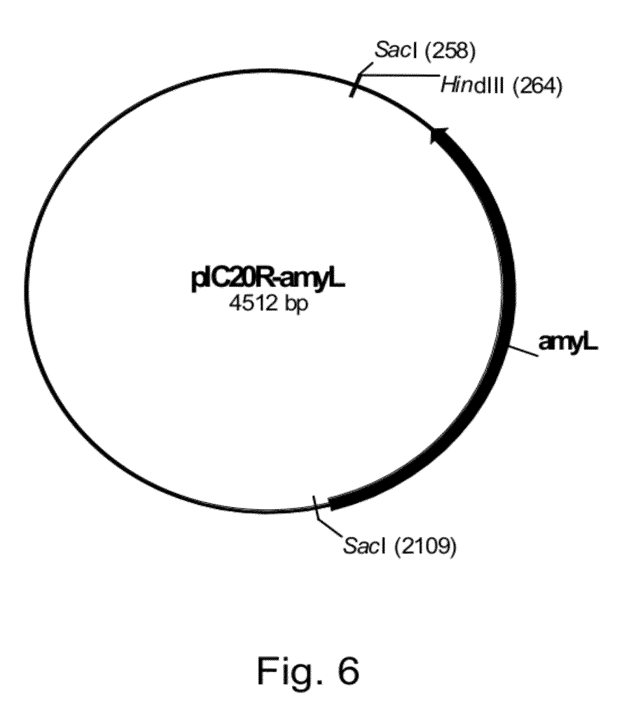

The purified amyL fragment and pIC20R vector fragment were ligated together using a Rapid DNA Ligation Kit and the ligation mix was transformed into E. coli DH5α™ competent cells (Invitrogen, Inc., Carlsbad, Calif., USA) according to the manufacturer's instructions. Transformants were selected on 2×YT agar plates supplemented with 100 μg of ampicillin per ml at 37° C. Plasmid DNA was purified from several transformants using a BIOROBOT® 9600 and analyzed by Hind III digestion. The digestions were resolved by 0.8% agarose gel electrophoresis using 0.5×TBE buffer. A plasmid was identified by the presence of a 4.5 kb Hind III fragment and designated pIC20R-amyL (FIG. 6).

Plasmids pIC20R-amyL and pHP13 ampMCS (U.S. Pat. No. 5,955,310) were digested with Sac I and Hind III. The digestions were each resolved by 0.8% agarose gel electrophoresis using 0.5×TBE buffer. An amyL fragment of approximately 1.8 kb from pIC20R-amyL and a vector fragment of approximately 5.2 kb from pHP13 ampMCS were excised from the gels and extracted using a QIAQUICK® Gel Extraction Kit.

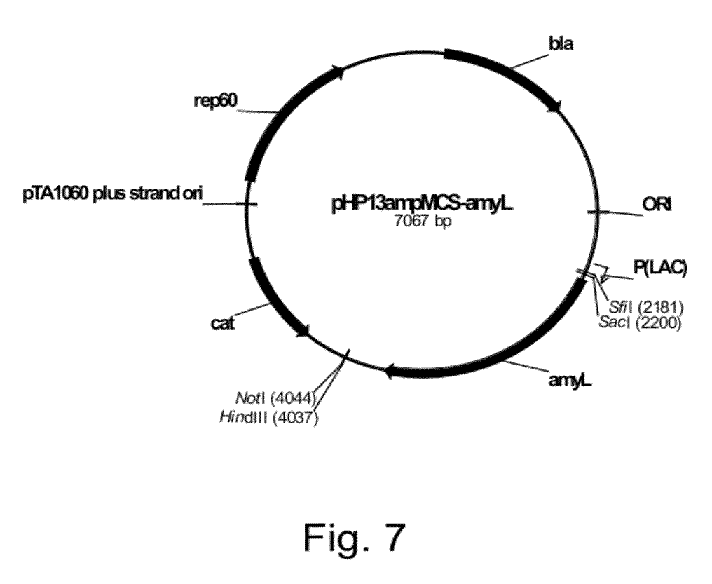

The purified amyL fragment and pHP13 ampMCS vector fragment were ligated together using a Rapid DNA Ligation Kit and the ligation mix was transformed into E. coli DH5α™ competent cells according to the manufacturer's instructions. Transformants were selected on 2×YT agar plates supplemented with 100 μg of ampicillin per ml at 37° C. Plasmids were purified from several transformants using a BIOROBOT® 9600 and analyzed by Sac I and Hind III digestion. The digestions were resolved by 0.8% agarose gel electrophoresis using 0.5×TBE buffer. A plasmid was identified by the presence of a 1.8 kb Sac I-Hind III fragment and designated pHP13 ampMCS-amyL (FIG. 7).

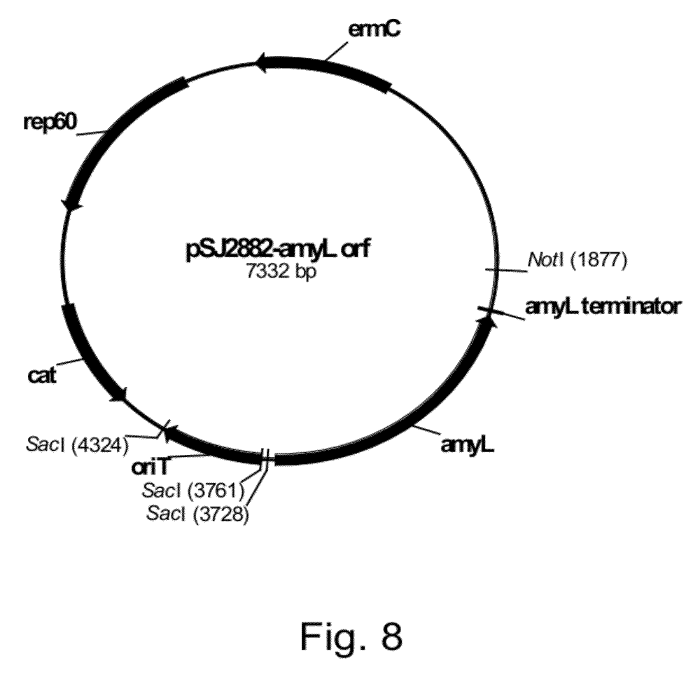

Plasmid pHP13 ampMCS-amyL and pSJ2882MCS (U.S. Pat. No. 5,891,701) were digested with Sfi I and Not I. The digestions were each resolved by 0.8% agarose gel electrophoresis using 0.5×TBE buffer. An amyL fragment of approximately 1.87 kb from pHP13 ampMCS-amyL and a vector fragment of approximately 5.4 kb from pSJ2882MCS were excised from the gels and extracted using a QIAQUICK® Gel Extraction Kit. The purified amyL fragment and pSJ2882 vector fragment were ligated together using a Rapid DNA Ligation Kit and the ligation mix was transformed into Bacillus subtilis PL1801 spoIIE competent cells (U.S. Pat. No. 5,955,310). Transformants were selected on TBAB agar plates supplemented with 6 μg of chloramphenicol per ml at 37° C. Plasmids were isolated using a BIOROBOT® 9600 and purified from several transformants using a QIAQUICK®DNA Purification Kit and analyzed by Sac I and Not I digestion. The digestions were resolved by 0.8% agarose gel electrophoresis using 0.5×TBE buffer. A plasmid was identified by the presence of a 1.87 kb Sac I-Not I fragment and designated pSJ2882-amyL orf (FIG. 8).

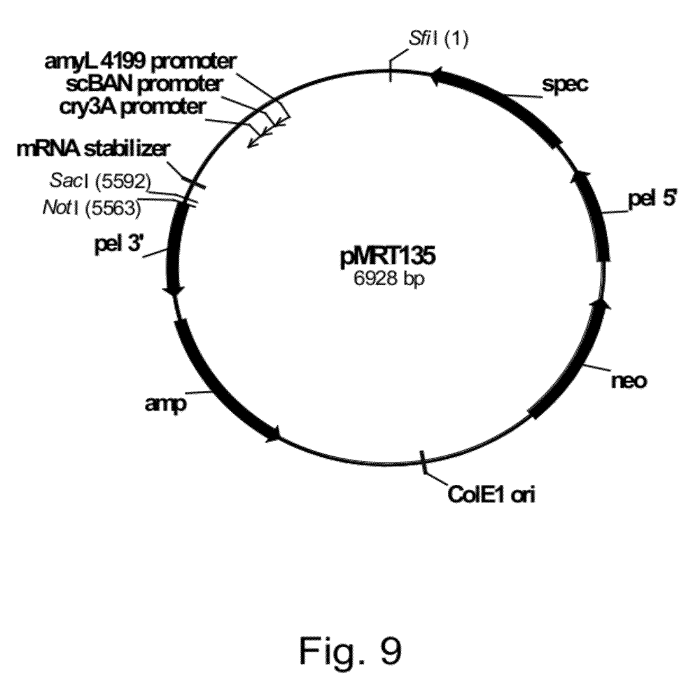

Plasmids pMDT100 and pRB165 (U.S. Patent Application 2008/0241887) were digested with Sfi I plus Sac I. The digestions were each resolved by 0.8% agarose gel electrophoresis using 0.5×TBE buffer. A promoter fragment of approximately 1.3 kb from pMDT100 and a vector fragment of approximately 5.6 kb from pRB165 were excised from the gels and extracted using a QIAQUICK® Gel Extraction Kit. The two purified fragments were ligated together using a Rapid DNA Ligation Kit and the ligation mix was transformed into E. coli SURE® competent cells according to the manufacturer's instructions. Transformants were selected on 2×YT agar plates supplemented with 100 μg of ampicillin per ml at 37° C. Plasmids were isolated and purified from several transformants using a BIOROBOT® 9600 and analyzed by Sfi I plus Sac I digestion. The digestions were resolved by 0.8% agarose gel electrophoresis using 0.5×TBE buffer. A plasmid was identified by the presence of an approximately 1.3 kb Sfi I/Sac I fragment and designated pMRT135 (FIG. 9).

Plasmids pSJ2882-amyLorf and pMRT135 were digested with Sac I and Not I. The digestions were each resolved by 0.8% agarose gel electrophoresis using 0.5×TBE buffer. An amyL fragment of approximately 1.85 kb from pSJ2882-amyL orf and a vector fragment of approximately 6.9 kb from pMRT135 were excised from the gels and extracted using a QIAQUICK® Gel Extraction Kit. The purified amyL fragment and pMRT135 vector fragment were ligated together using a Rapid DNA Ligation Kit and the ligation mix was transformed into Bacillus subtilis 168Δ4 competent cells. Transformants were selected on TBAB agar plates overlayed with 8 ml of 1% starch azure dye (Sigma Chemical Co., St. Louis, Mo., USA)-1.5% Bacto agar supplemented with 120 μg of spectinomycin per ml and incubated overnight at 37° C. A halo surrounding a patch was indicative of the amyL gene being over-expressed. Two transformant colonies were patched onto a TBAB agar plate supplemented with 5 μg of neomycin per ml to verify neomycin sensitivity. One such transformant was chosen and designated Bacillus subtilis MATA28.

Plasmids pSJ2882-amyLorf and pMDT100 were digested with Sac I and Not I. The digestions were each resolved by 0.8% agarose gel electrophoresis using 0.5×TBE buffer. An amyL fragment of approximately 1.85 kb from pSJ2882-amyLorf and a vector fragment of approximately 8 kb from pMDT100 were excised from the gels and extracted using a QIAQUICK® Gel Extraction Kit. The purified amyL fragment and pMDT100 vector fragment were ligated together using a Rapid DNA Ligation Kit and the ligation mix was transformed into B. subtilis MATA28 competent cells. Chloramphenicol resistant transformants were selected at 37° C. after 16 hours of growth on TBAB plates supplemented with 5 μg of chloramphenicol per ml. To screen for integration of the plasmid by double cross-over at the amyE locus, Bacillus subtilis primary transformants were patched onto TBAB plates supplemented with 6 μg of neomycin per ml and onto TBAB plates supplemented with 5 μg of chloramphenicol per ml. Integration of the plasmid by double cross-over at the amyE locus does not incorporate the neomycin resistance gene and therefore renders the strain neomycin sensitive. A chloramphenicol resistant, neomycin sensitive transformant was identified and designated Bacillus subtilis MATA31.

### Example 6

### Construction of Bacillus subtilis BW223

Plasmid pBW222 was constructed by splicing by overlap extension (SOE) (Horton et al., 1989, supra) to generate and fuse a hybrid amyL/amyQ signal sequence to the synthetic mature region of the Family 11 xylanase from Dictyoglomus thermophilum. The amyL signal sequence and promoter region were PCR amplified from Bacillus subtilis strain MATA31 chromosomal DNA using the primer pair 065452/065481 shown below. Chromosomal DNA was obtained according to the procedure of Pitcher et al., 1989, supra.

Primer 065452:
(SEQ ID NO: 28)
5′-TATCAATTGGTAACTGTATC-3′
Primer 065481:
(SEQ ID NO: 29)
5′-CGGCAAACTGACAAATAACAGCGTGCACATAAGCACCAATCGGGCG
TAAAGCCG-3′

Underlined letters represent amyQ signal sequence while bold letters represent amyL signal sequence.

The mature xylanase sequence region was PCR amplified from Bacillus subtilis strain SMO59 chromosomal DNA using the primer pair 065480/065520, shown below. Chromosomal DNA was obtained according to the procedure of Pitcher et al., 1989, supra.

Primer 065480:
(SEQ ID NO: 30)
5′-CTGTTATTTGTCAGTTTGCCGATTACAAAAACATCAGCCCAGACAT
CAATCACACTTAC-3′
Primer 065520:
(SEQ ID NO: 31)
5′-CCTTTGCGGCTTTTTGCATC-3′

Underlined letters represent amyQ signal sequence while bold letters represent Dictyoglomus thermophilum xylanase coding sequence.

The amplifications above were conducted in 50 μl reactions composed of 10 ng of chromosomal DNA, 1.0 μM of each primer, 200 μM each of dATP, dCTP, dGTP, and dTTP, 1× ThermoPol buffer (New England Biolabs, Inc., Ipswich, Mass., USA), and 2.5 units of Taq DNA polymerase (New England Biolabs, Inc., Ipswich, Mass., USA). The amplifications were performed in a ROBOCYCLER® 40 Temperature Cycler programmed for 1 cycle at 95° C. for 2 minutes; 30 cycles each at 95° C. for 1 minute, 53° C. for 1 minute, and 72° C. for 2 minutes; and 1 cycle at 72° C. for 7 minutes. PCR products were resolved by 0.8% agarose gel electrophoresis using 0.5×TBE buffer. A band of approximately 1.0 kb obtained using the primer pair 065452/065481 for the amyL promoter and signal sequence and a band of approximately 1.0 kb obtained using the primer pair 065480/065520 for the mature xylanase coding sequence were excised from the gels and extracted using a QIAQUICK® Gel Extraction Kit.

The final SOE fragment was amplified using primer 065455 shown below and primer 065520 shown above using the following PCR conditions. The amplification was conducted in a 50 μl reaction composed of 10 ng of each PCR fragment, 200 μM each of dATP, dCTP, dGTP, and dTTP, 1× ThermoPol buffer, and 2.5 units of Taq DNA polymerase. The amplifications were performed in a ROBOCYCLER® 40 Temperature Cycler programmed for 1 cycle at 95° C. for 1 minutes; and 4 cycles each at 95° C. for 1 minute, 52° C. for 1 minute, and 72° C. for 2 minutes. At this point 1.0 μM of each primer was added to the reaction followed by 26 cycles each at 95° C. for 1 minute, 55° C. for 1 minute, and 72° C. for 2 minutes; and 1 cycle at 72° C. for 3 minutes. The PCR product was resolved by 0.8% agarose gel electrophoresis using 0.5×TBE buffer. A band of approximately 1.5 kb corresponding to the SOE fragment was excised from the gel and extracted using a QIAQUICK® Gel Extraction Kit.

Primer 065455:
(SEQ ID NO: 32)
5′-CAGATTACAAATATATTCGG-3′

The SOE fragment of approximately 1.5 kb was cloned into pCR®2.1 using a TA-TOPO® Cloning Kit according to the manufacturer's instructions and transformed into ONE SHOT® TOP10 Chemically Competent E. coli cells according to the manufacturer's instructions. Transformants were selected on 2×YT agar plates supplemented with 100 μg of ampicillin per ml and incubated at 37° C. for 16 hours. The DNA sequence of the cloned fragment was verified by DNA sequencing with M13 forward and reverse primers, and internal primers 065455 and 065520 shown above and primers 065458 and 065459 shown below. The plasmid was designated pBW222.

Primer 065458:
(SEQ ID NO: 33)
5′-GTAGATGTCATATGTGCCA-3′
Primer 065459:
(SEQ ID NO: 34)
5′-TGGCACATATGACATCTAC-3′

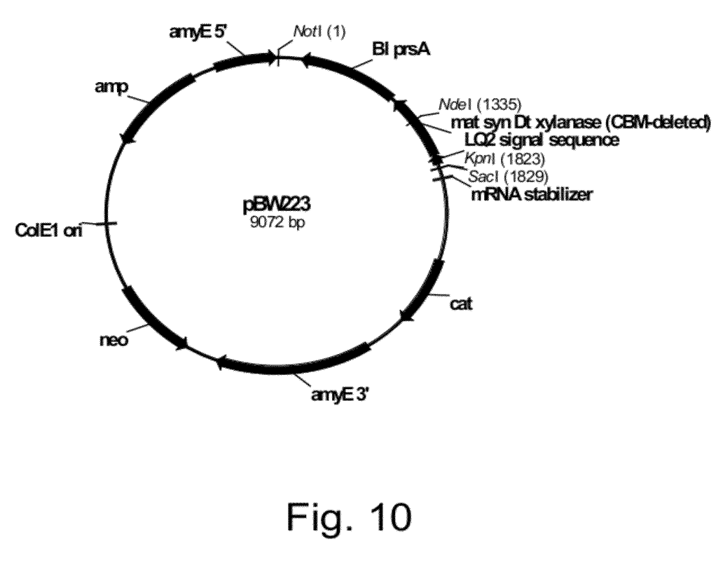

Plasmids pBW222 and pSMO280 were digested with Sac I and Nde I. The digestions were each resolved by 0.8% agarose gel electrophoresis using 0.5×TBE buffer. A fragment of approximately 0.5 kb from pBW222 and a vector fragment of approximately 8.6 kb from pSMO280 were excised from the gels and extracted using a QIAQUICK® Gel Extraction Kit. The two purified fragments were ligated together using a Rapid DNA Ligation Kit and the ligation mix was transformed into E. coli SURE® competent cells according to the manufacturer's instructions. Transformants were selected on 2×YT agar plates supplemented with 100 μg of ampicillin per ml at 37° C. Plasmids were isolated using a BIOROBOT® 9600 from several transformants and analyzed by Kpn I digestion. The digestions were resolved by 0.8% agarose gel electrophoresis using 0.5×TBE buffer. A plasmid was identified by the presence of an approximately 9 kb Kpn I fragment and designated pBW223 (FIG. 10).

Plasmids pBW223 and pMDT100 were digested with Sac I and Not I. The digestions were each resolved by 0.8% agarose gel electrophoresis using 0.5×TBE buffer. A xylanase fragment of approximately 1.8 kb from pBW223 and a vector fragment of approximately 8.0 kb from pMDT100 were excised from the gels and extracted using a QIAQUICK® Gel Extraction Kit. The two purified fragments were ligated together using a Rapid DNA Ligation Kit and the ligation mix was transformed into Bacillus subtilis 168Δ4 competent cells. Chloramphenicol resistant transformants were selected at 37° C. after 16 hours of growth on TBAB plates supplemented with 5 μg of chloramphenicol per ml. To screen for integration of the plasmid by double cross-over at the amyE locus, Bacillus subtilis primary transformants were patched onto TBAB plates supplemented with 6 μg of neomycin per ml and onto TBAB plates supplemented with 5 μg of chloramphenicol per ml. Integration of the plasmid by double cross-over at the amyE locus does not incorporate the neomycin resistance gene and therefore renders the strain neomycin sensitive. A chloramphenicol resistant, neomycin sensitive transformant was identified which harbored the triple promoter/amyL-amyQ hybrid sig seq/Dt xylanase/prsA expression cassette in the amyE locus and designated Bacillus subtilis 168Δ4 amyE::triple promoter/amyL-amyQ sig seq/Dt xyl/prsA.

Chromosomal DNA was purified from Bacillus subtilis 168Δ4 amyE::triple promoter/amyL-amyQ sig seq/Dt xyl/prsA according to the Pitcher et al., 1989, supra, and 0.1 μg was transformed into competent Bacillus subtilis SMO25 cells. Chloramphenicol resistant transformants were selected at 37° C. after 16 hours of growth on TBAB plates supplemented with 5 μg of chloramphenicol per ml. A chloramphenicol resistant transformant was chosen, single colony purified, and designated Bacillus subtilis BW223.

### Example 7

### Construction of Bacillus subtilis BW229

The Bacillus subtilis thiol-disulfide oxidoreductase genes bdbC (SEQ ID NO: 1 [DNA sequence] and SEQ ID NO: 2 [deduced amino acid sequence]) and bdbD (SEQ ID NO: 3 [DNA sequence] and SEQ ID NO: 4 [deduced amino acid sequence]) were deleted in Bacillus subtilis BW223 to test whether deleting the two genes would enhance production of the Dictyoglomus thermophilum Family 11 xylanase. Both of the genes are contained within an operon in Bacillus subtilis and encode thiol-disulfide oxidoreductases involved in forming disulfide bonds in secreted proteins in B. subtilis. A DNA fragment containing the deletion of the two genes was generated by splicing by overlap extension (SOE) (Horton et al., 1989, supra).

The bdbD gene promoter region and bdbC gene downstream sequence were PCR amplified from Bacillus subtilis strain A164 (U.S. Pat. No. 5,698,415) chromosomal DNA using primer pairs 066467/066468 and 066469/066470, respectively, shown below. Chromosomal DNA was obtained according to the procedure of Pitcher et al., 1989, supra.

Primer 066467:
(SEQ ID NO: 35)
5′-GGATCCGCGATGGGAGGCCTTGGCTC-3′
Primer 066468:
(SEQ ID NO: 36)
5′-CCCGGGTTCACTCCGACACCTCATCG-3′
Primer 066469:
(SEQ ID NO: 37)
5′-CGATGAGGTGTCGGAGTGAACCCGGGTATGTGCCTGCTGAAAGGC
G-3′
Primer 066470:
(SEQ ID NO: 38)
5′-GGTACCTCCCTGCTCCCATAGCGCTG-3′

PCR amplifications for each of the primer pairs above were conducted in 50 μl composed of 10 ng of chromosomal DNA, 1.0 μM of each primer, 200 μM each of dATP, dCTP, dGTP, and dTTP, 1× ThermoPol buffer with 2.5 mM MgCl₂, and 2.5 units of Taq DNA polymerase. The reactions were performed in a ROBOCYCLER® 40 Temperature Cycler programmed for 1 cycle at 95° C. for 2 minutes; 30 cycles each at 95° C. for 1 minute, 53° C. for 1 minute, and 72° C. for 1 minute; and 1 cycle at 72° C. for 7 minutes. The PCR products were each resolved by 0.8% agarose gel electrophoresis using 0.5×TBE buffer. A band of approximately 400 bp obtained using the primer pair 066467/066468 for the bdbD gene promoter region and a band of approximately 400 bp obtained using the primer pair 066469/066470 for the bdbC downstream region were excised from the gels and extracted using a QIAQUICK® Gel Extraction Kit.

The final SOE fragment was amplified using primer 066467 and primer 066470, shown above, under the same PCR conditions described above. The PCR products were resolved by 0.8% agarose gel electrophoresis using 0.5×TBE buffer. A band of approximately 800 bp obtained for the final SOE fragment was excised from the gel and extracted using a QIAQUICK® Gel Extraction Kit.

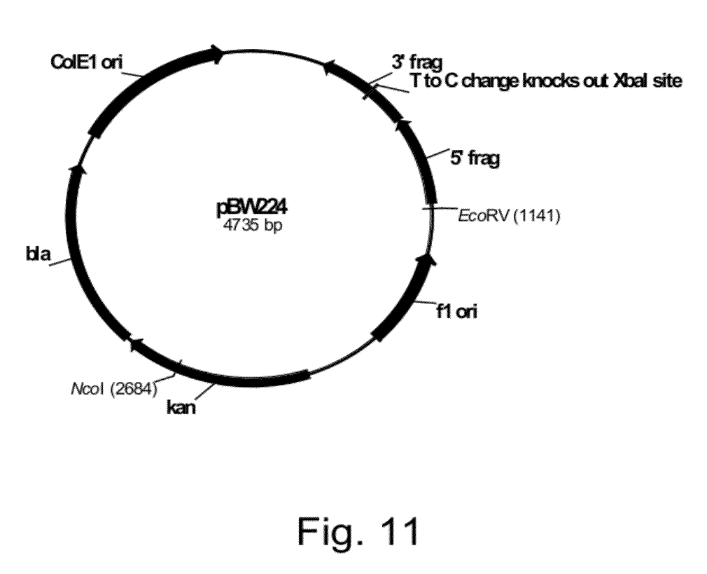

The 800 bp SOE fragment was cloned into pCR®2.1 using a TA-TOPO® Cloning Kit according to the manufacturer's instructions and transformed into ONE SHOT® TOP10 Chemically Competent E. coli cells according to the manufacturer's instructions. Transformants were selected on 2×YT agar plates supplemented with 100 μg of ampicillin per ml and incubated at 37° C. for 16 hours. Plasmid DNA was isolated from several transformants using a BIOROBOT® 9600 and submitted to DNA sequencing with M13 forward and reverse primers. All contained at least one base pair change in the promoter region of the yvgT gene (just downstream of the bdbC gene). A plasmid was identified that had only one base pair change (the first T of the Xba I site located in that region) and was designated pBW224 (FIG. 11).

The kanamycin resistance marker of plasmid pBW224 was inactivated as follows. Plasmid pBW224 was digested with Nco I and the ends were blunted by incubation for 20 minutes at 11° C. with T4 DNA polymerase and 25 μM each of dATP, dCTP, dGTP, and dTTP, followed by heat-inactivation of the T4 DNA polymerase by incubation for 10 minutes at 75° C. The linearized plasmid was then ligated together using a Rapid DNA Ligation Kit and the ligation mix was transformed into E. coli SURE® competent cells according to the manufacturer's instructions. Transformants were selected on 2×YT agar plates supplemented with 100 μg of ampicillin per ml at 37° C. Plasmids were isolated using a BIOROBOT® 9600 and analyzed by Nco I plus Eco RV digestion. The digestions were resolved by 0.8% agarose gel electrophoresis using 0.5×TBE buffer. A plasmid was identified by the presence of a 4.7 kb Nco I/Eco RV fragment and designated pBW225.

Plasmid pBEST501 Maya et al., 1989, Nucleic Acids Res. 17: 4410) was digested with Sma I. The digestion was resolved by 0.8% agarose gel electrophoresis using 0.5×TBE buffer. A fragment of approximately 1.36 kb containing a neomycin resistance marker was excised from the gel and extracted using a QIAQUICK® Gel Extraction Kit.

Plasmid pBW225 was digested with Sma I. The digestion was resolved by 0.8% agarose gel electrophoresis using 0.5×TBE buffer. A vector fragment of approximately 4.7 kb was excised from the gel and extracted using a QIAQUICK® Gel Extraction Kit.

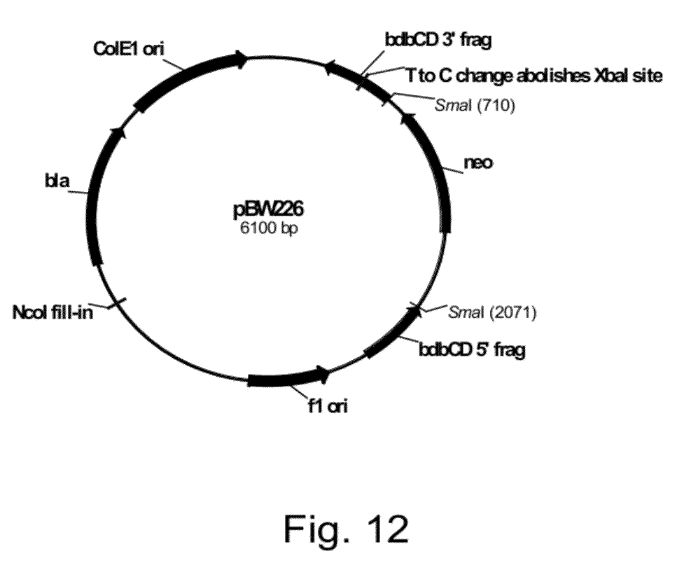

The purified neomycin resistance marker fragment and the pBW225 vector fragment were ligated together using a Rapid DNA Ligation Kit and the ligation mix was transformed into E. coli XL-1 Blue competent cells according to the manufacturer's instructions. Transformants were selected on 2×YT agar plates supplemented with 50 μg of kanamycin per ml at 37° C. Plasmids were isolated using a BIOROBOT® 9600 and analyzed by Sma I digestion. The digestions were resolved by 0.8% agarose gel electrophoresis using 0.5×TBE buffer gel. A plasmid harboring the neomycin resistance gene was identified by the presence of a 1.36 kb Sma I fragment and designated pBW226 (FIG. 12).

In order to disrupt the bdbC and bdbD genes, plasmid pBW226 (1 μg of supercoiled DNA) was transformed into Bacillus subtilis 168Δ4 competent cells. Neomycin resistant transformants were selected at 37° C. and obtained after 16 hours of growth on TBAB plates supplemented with 5 μg of neomycin per ml. To confirm the desired disruption, chromosomal DNA was isolated from 5 transformants according to Pitcher et al., 1989, supra, and analyzed by PCR using primers 066467 and 066470, shown above.

PCR amplifications were conducted in 50 μl reactions composed of 10 ng of chromosomal DNA, 0.4 μM of each primer, 200 μM each of dATP, dCTP, dGTP, and dTTP, 1×PCR Buffer II with 2.5 mM MgCl₂, and 2.5 units of AmpliTaq GOLD® DNA Polymerase. The reactions were performed in a ROBOCYCLER® 40 Temperature Cycler programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. The PCR products were resolved by 0.8% agarose gel electrophoresis using 0.5×TBE buffer. A fragment of approximately 2.2 kb was observed in 3 of the 5 transformants.

The three transformants were analyzed further by performing a second PCR amplification using primer pair 066469/066470 shown above to determine whether the aforementioned base pair change at the Xba I site had been incorporated into the chromosome during the disruption of the bdbC and bdbD genes.

The PCR amplifications were conducted in 50 μl reactions composed of 10 ng of chromosomal DNA, 0.4 μM of each primer, 200 μM each of dATP, dCTP, dGTP, and dTTP, 1×PCR Buffer II with 2.5 mM MgCl₂, and 2.5 units of AmpliTaq GOLD® DNA Polymerase. The reactions were performed in a ROBOCYCLER® 40 Temperature Cycler programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. The PCR products were resolved by 0.8% agarose gel electrophoresis using 0.5×TBE buffer. As expected, bands of approximately 400 bp were observed. The PCR fragments were excised from the gels and extracted using a QIAQUICK® DNA Purification Kit. The purified fragments were sequenced using primers 066469 and 066470, shown above. One of the three strains contained the wild-type sequence (the Xba I site was intact) and was designated Bacillus subtilis 168Δ4 bdbCDΔNeo.

Chromosomal DNA was isolated from Bacillus subtilis 168Δ4 bdbCDΔNeo (Pitcher et al., 1989, supra) and 0.5 μg was transformed into competent Bacillus subtilis BW223. Neomycin resistant transformants were selected at 37° C. after 16 hours of growth on TBAB plates supplemented with 5 μg of neomycin per ml. One transformant was single colony purified and designated Bacillus subtilis BW229.

### Example 8

### Construction of Bacillus subtilis Strain BW230

The thiol-disulfide oxidoreductase genes bdbA (SEQ ID NO: 39 [DNA sequence] and SEQ ID NO: 40 [deduced amino acid sequence]) and bdbB (SEQ ID NO:41 [DNA sequence] and SEQ ID NO: 42 [deduced amino acid sequence]) were deleted in Bacillus subtilis BW223 to test whether deleting the two genes would enhance production of the Dictyoglomus thermophilum Family 11 xylanase. Both of the genes are contained within an operon in Bacillus subtilis. A DNA fragment containing the deletion of the two genes was generated by the SOE method where the following three PCR fragments were fused together: a 3 kb fragment (fragment A) that contains DNA sequence located just upstream of the bdbA gene, a 1.2 kb fragment (fragment B) that contains a spectinomycin resistance gene from plasmid pSJ5218 (U.S. Patent Application 2003/0032186), and a 3 kb fragment (fragment C) that contains DNA sequence located just downstream of the bdbB gene.

Fragment A was amplified from Bacillus subtilis strain A164 (U.S. Pat. No. 5,698,415) chromosomal DNA using primer pair 067207/067208 shown below. Chromosomal DNA was obtained according to the procedure of Pitcher et al., 1989, supra.

Primer 067207:
(SEQ ID NO: 43)
5′-CAATGGATTCGCAGGTATTAGATG-3′
Primer 067208:
(SEQ ID NO: 44)
5′ CATCCTTTCACAATTTGTCTACAGCTGAGCTTTTCCTAATCCACTA
CC 3′

The amplifications were composed of 10 μl of 5× PHUSION™ HF buffer (New England Biolabs, Inc., Ipswich, Mass., USA), 1 μl of 10 mM dNT mix, 1 μl (50 pMoles) of primer 067207, 1 μl (50 pMoles) of primer 067208, 1 μl of template DNA (10 ng of chromosomal DNA), 0.5 μl of PHUSION™ DNA polymerase (New England Biolabs, Inc., Ipswich, Mass., USA), and 35 μl of sterile distilled water. The amplifications were performed with a PTC-200 Peltier Thermal Cycler (MJ Research, Inc., Waltham, Mass., USA) programmed for 1 cycle at 96° C. for 2 minutes; 11 cycles each at 94° C. for 30 seconds, 60° C. for 45 seconds and subtracting 1° C. after each cycle, and 72° C. for 2 minutes; and 20 cycles each at 94° C. for 30 seconds, 50° C. for 45 seconds, and 72° C. for 2 minutes and adding 20 seconds after each cycle; and 1 cycle at 72° C. for 5 minutes. The PCR product was resolved by 0.8% agarose gel electrophoresis using 0.5×TBE buffer. A band of approximately 3 kb was excised from the gel and extracted using a QIAQUICK® Gel Extraction Kit.

Fragment B was amplified from pSJ5218 using primer pair 067209/067210 shown below.

Primer 067209:
(SEQ ID NO: 45)
5′-GGTAGTGGATTAGGAAAAGCTCAGCTGTAGACAAATTGTGAAAGGA
TG-3′
Primer 067210:
(SEQ ID NO: 46)
5′-CCCTCTTACAAGGCGGGTTACTTCCAAGTGTTCGCTTCGCTCTCAC
TG-3′

The amplifications were composed of 10 μl of 5× PHUSION™ HF buffer, 1 μl of 10 mM dNT mix, 1 μl (50 pMoles) of primer 067209, 1 μl (50 pMoles) of primer 067210, 1 μl of template DNA (1 ng of plasmid DNA), 0.5 μl of PHUSION™ DNA polymerase, and 35 μl of sterile distilled water. The amplifications were performed with a PTC-200 Peltier Thermal Cycler programmed for 1 cycle at 94° C. for 3 minutes; 30 cycles each at 94° C. for 1 minute, 55° C. for 1 minute, and 72° C. for 2 minutes; and 1 cycle at 72° C. for 7 minutes. The PCR product was resolved by 0.8% agarose gel electrophoresis using 0.5×TBE buffer. A band of approximately 1.2 kb was excised from the gel and extracted using a QIAQUICK® Gel Extraction Kit.

Fragment C was amplified from Bacillus subtilis strain A164 (U.S. Pat. No. 5,698,415) chromosomal DNA using primer pair 067211/067212 shown below. Chromosomal DNA was obtained according to the procedure of Pitcher et al., 1989, supra.

Primer 067211:
(SEQ ID NO: 47)
5′-CAGTGAGAGCGAAGCGAACACTTGGAAGTAACCCGCCTTGTAAGAG
GG-3′
Primer 067212:
(SEQ ID NO: 48)
5′-AAGACGAGTGTCGGGTAACGTAGG-3′

The amplifications were composed of 10 μl of 5× PHUSION™ HF buffer, 1 μl of 10 mM dNT mix, 1 μl (50 pMoles) of primer 067211, 1 μl (50 pMoles) of primer 067212, 1 μl of template DNA (10 ng of chromosomal DNA), 0.5 μl of PHUSION™ DNA polymerase, and 35 μl of sterile distilled water. The amplifications were performed with a PTC-200 Peltier Thermal Cycler programmed for 1 cycle at 94° C. for 3 minutes; 30 cycles each at 94° C. for 1 minute, 55° C. for 1 minute, and 72° C. for 2 minutes; and 1 cycle at 72° C. for 7 minutes. The PCR product was resolved by 0.8% agarose gel electrophoresis using 0.5×TBE buffer. A band of approximately 3 kb was excised from the gel and extracted using a QIAQUICK® Gel Extraction Kit.

The final SOE fragment was amplified using primer 067207 and primer 067212. The amplifications were composed of 10 μl of 5× PHUSION™ HF buffer, 1 μl of 10 mM dNTP mix, 126 ng of fragment A, 48 ng of fragment B, 126 ng of fragment C, 1 μl (50 pMoles) of primer 067207, 1 μl (50 pMoles) of primer 067212, 0.5 μl of PHUSION™ DNA polymerase, and sterile distilled water to a final volume to 50 μl. The amplifications were performed with a PTC-200 Peltier Thermal Cycler programmed for 1 cycle at 96° C. for 2 minutes; 11 cycles each at 94° C. for 30 seconds, 60° C. for 45 seconds and subtracting 1° C. after each cycle, and 72° C. for 4 minutes; and 20 cycles each at 94° C. for 30 seconds, 50° C. for 45 seconds, and 72° C. for 4 minutes and adding 20 seconds after each cycle; and 1 cycle at 72° C. for 5 minutes. A PCR product of approximately 7.5 kb was resolved by 0.8% agarose gel electrophoresis using 0.5×TBE buffer. Then 5 μl of the SOE fragment was transformed into competent Bacillus subtilis BW223. Spectinomycin resistant transformants were selected at 37° C. after 16 hours of growth on TBAB plates supplemented with 120 μg of spectinomycin per ml. One transformant was single colony purified and designated Bacillus subtilis BW230.

### Example 9

### Construction of Bacillus subtilis Strain BW231

All 4 bdb genes (bdbA, bdbB, bdbC, and bdbD) were deleted in Bacillus subtilis strain BW223. Chromosomal DNA was obtained from Bacillus subtilis strain BW229 and 10 ng was transformed into competent Bacillus subtilis BW230. Neomycin resistant transformants were selected at 37° C. after 16 hours of growth on TBAB plates supplemented with 5 μg of neomycin per ml. One transformant was single colony purified and designated Bacillus subtilis BW231.

### Example 10

### Fermentations of Bacillus subtilis Strains BW223, BW229, BW230, and BW231

Each of the Bacillus subtilis strains designated BW223, BW229 (ΔbdbCD), BW230 (ΔbdbAB), and BW231 (ΔbdbABCD) were streaked onto agar slants and incubated for about 24 hours at 37° C. The agar medium was composed of 10 g of soy peptone, 10 g of sucrose, 2 g of trisodium citrate dihydrate, 4 g of KH₂PO₄, 5 g of Na₂HPO₄, 15 g of Bacto agar, 0.15 mg of biotin, 2 ml of trace metals solution, and deionized water to 1 liter. The trace metals solution was composed of 1.59 g of ZnSO₄.7H₂O, 0.76 g of CuSO₄.5H₂O, 7.52 g of FeSO₄.7H₂O, 1.88 g of MnSO₄.H₂O, 20 g of citric acid, and deionized water to 1 liter. Approximately 15 ml of sterile buffer (7.0 g of Na₂HPO₄, 3.0 g of KH₂PO₄, 4.0 g of NaCl, 0.2 g of MgSO₄.7H₂O, and deionized water to 1 liter) were used to gently wash off some of the cells from the agar surface. The bacterial suspensions were then each inoculated into baffled shake flasks containing 100 ml of growth medium composed of 11 g of soy bean meal, 0.4 g of Na₂HPO₄, 5 drops of antifoam, and deionized water to 100 ml. The inoculated shake flasks were incubated at 37° C. for about 20 hours with shaking at 300 rpm, after which 100 ml (obtained by combining the media from two independent shake flasks with the same strain) were used for inoculation of a 3 liter fermentor containing 900 ml of medium composed of 40 g of hydrolyzed potato protein, 6 g of K₂SO₄, 4 g of Na₂HPO₄, 12 g of K₂HPO₄, 4 g of (NH₄)₂SO₄, 0.5 g of CaCO₃, 2 g of citric acid, 4 g of MgSO₄, 40 ml of trace metals solution (described above), 1 mg of biotin (biotin was added as 1 ml of a 1 g per liter biotin solution in the buffer described above), 1.3 ml of antifoam, and deionized water to 900 ml. The medium was adjusted to pH 5.25 with phosphoric acid prior to being autoclaved.

The fermentation was carried out as a fedbatch fermentation with sucrose solution being the feed. The fermentation temperature was held constant at 37° C. The tanks were aerated with 3 liter air per minute, and the agitation rate was held in the range of 1,500-1,800 rpm. The fermentation time was around 60-70 hours. The pH was maintained in the range of pH 6.5-7.3.

The fermentations were assayed for xylanase activity according to the following procedure. Culture supernatants were diluted appropriately in 0.1 M sodium acetate pH 5.0. A purified Dictyoglomus thermophilum xylanase as a standard was diluted using 2-fold steps starting with a 1.71 μg/ml concentration and ending with a 0.03 μg/ml concentration in 0.1 M sodium acetate pH 5.0. A total of 40 μl of each dilution including standard were transferred to a 96-well flat bottom plate. Using a BIOMEK® NX (Beckman Coulter, Fullerton Calif., USA), a 96-well pippetting workstation, 40 μl an Azo-Wheat arabinoxylan (Megazyme International, Ireland) substrate solution (1% w/v) was added to each well and then incubated at 50° C. for 30 minutes. Upon completion of the incubation the reaction was stopped with 200 μl of ethanol (95% v/v). The samples were then incubated at ambient temperatures for 5 minutes followed by centrifugation at 3,000 rpm for 10 minutes. A 150 μl volume of each supernatant was removed using the BIOMEK® NX and dispensed into a new 96-well flat bottom plate. The optical density of 590 nm was measured using a SPECTRAMAX® 250 plate reader (Molecular Devices, Sunnyvale Calif., USA). Sample concentrations were determined by extrapolation from the generated standard curve.

The results as shown in Table 2 demonstrated that deletion of the bdbC and bdbD genes increased the relative yield of the xylanase by 55%.

TABLE 2

| | | Relative Xylanase |
|---|---|---|
| B. subtilis strain | Description | Yield |
| BW223 | | 100 |
| BW229 | ΔbdbCD | 155 |
| BW230 | ΔbdbAB | 87 |
| BW231 | ΔbdbABCD | 108 |

The present invention is furthered described by the following numbered paragraphs:

[1] An isolated mutant of a parent bacterial cell, comprising a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, wherein the mutant cell is deficient in production of the thiol-disulfide oxidoreductase compared to the parent bacterial cell when cultivated under the same conditions.

[2] The mutant of paragraph 1, wherein the thiol-disulfide oxidoreductase gene is selected from the group consisting of: (a) a gene encoding a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof; (b) a gene encoding a thiol-disulfide oxidoreductase comprising a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; and (c) a gene encoding a thiol-disulfide oxidoreductase comprising a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[3] The mutant of paragraph 2, wherein the thiol-disulfide oxidoreductase gene encodes a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[4] The mutant of paragraph 3, wherein the thiol-disulfide oxidoreductase gene encodes a thiol-disulfide oxidoreductase comprising an amino acid sequence having at least 65% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[5] The mutant of paragraph 4, wherein the thiol-disulfide oxidoreductase gene encodes a thiol-disulfide oxidoreductase comprising an amino acid sequence having at least 70% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[6] The mutant of paragraph 5, wherein the thiol-disulfide oxidoreductase gene encodes a thiol-disulfide oxidoreductase comprising an amino acid sequence having at least 75% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[7] The mutant of paragraph 6, wherein the thiol-disulfide oxidoreductase gene encodes a thiol-disulfide oxidoreductase comprising an amino acid sequence having at least 80% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[8] The mutant of paragraph 7, wherein the thiol-disulfide oxidoreductase gene encodes a thiol-disulfide oxidoreductase comprising an amino acid sequence having at least 85% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[9] The mutant of paragraph 8, wherein the thiol-disulfide oxidoreductase gene encodes a thiol-disulfide oxidoreductase comprising an amino acid sequence having at least 90% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[10] The mutant of paragraph 9, wherein the thiol-disulfide oxidoreductase gene encodes a thiol-disulfide oxidoreductase comprising an amino acid sequence having at least 95% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[11] The mutant of paragraph 10, wherein the thiol-disulfide oxidoreductase gene encodes a thiol-disulfide oxidoreductase comprising an amino acid sequence having at least 97% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[12] The mutant of paragraph 11, wherein the thiol-disulfide oxidoreductase gene encodes a thiol-disulfide oxidoreductase comprising an amino acid sequence having at least 99% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[13] The mutant of paragraph 2, wherein the thiol-disulfide oxidoreductase gene comprises a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[14] The mutant of paragraph 13, wherein the thiol-disulfide oxidoreductase gene comprises a nucleotide sequence that hybridizes under at least medium stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[15] The mutant of paragraph 14, wherein the thiol-disulfide oxidoreductase gene comprises a nucleotide sequence that hybridizes under at least medium-high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[16] The mutant of paragraph 15, wherein the thiol-disulfide oxidoreductase gene comprises a nucleotide sequence that hybridizes under at least high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[17] The mutant of paragraph 16, wherein the thiol-disulfide oxidoreductase gene comprises a nucleotide sequence that hybridizes under at least very high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[18] The mutant of paragraph 2, wherein the thiol-disulfide oxidoreductase gene comprises a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[19] The mutant of paragraph 18, wherein the thiol-disulfide oxidoreductase gene comprises a nucleotide sequence having at least 65% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[20] The mutant of paragraph 19, wherein the thiol-disulfide oxidoreductase gene comprises a nucleotide sequence having at least 70% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[21] The mutant of paragraph 20, wherein the thiol-disulfide oxidoreductase gene comprises a nucleotide sequence having at least 75% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[22] The mutant of paragraph 21, wherein the thiol-disulfide oxidoreductase gene comprises a nucleotide sequence having at least 80% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[23] The mutant of paragraph 22, wherein the thiol-disulfide oxidoreductase gene comprises a nucleotide sequence having at least 85% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[24] The mutant of paragraph 23, wherein the thiol-disulfide oxidoreductase gene comprises a nucleotide sequence having at least 90% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[25] The mutant of paragraph 24, wherein the thiol-disulfide oxidoreductase gene comprises a nucleotide sequence having at least 95% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[26] The mutant of paragraph 25, wherein the thiol-disulfide oxidoreductase gene comprises a nucleotide sequence having at least 97% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[27] The mutant of paragraph 26, wherein the thiol-disulfide oxidoreductase gene comprises a nucleotide sequence having at least 99% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[28] The mutant of paragraph 1, wherein the thiol-disulfide oxidoreductase gene encodes the thiol-disulfide oxidoreductase of SEQ ID NO: 2, SEQ ID NO: 50, SEQ ID NO: 52, SEQ ID NO: 54, SEQ ID NO: 56, SEQ ID NO: 58, or SEQ ID NO: 60.

[29] The mutant of paragraph 1, wherein the thiol-disulfide oxidoreductase gene encodes the thiol-disulfide oxidoreductase of SEQ ID NO: 4, SEQ ID NO: 62, SEQ ID NO: 64, SEQ ID NO: 66, or SEQ ID NO: 68.

[30] The mutant of any of paragraphs 1-29, wherein the polypeptide encoded by the first polynucleotide is an antigen, an enzyme, a growth factor, a hormone, an immunodilator, a neurotransmitter, a receptor, a reporter protein, a structural protein, or a transcription factor.

[31] The mutant of paragraph 30, wherein the enzyme is an oxidoreductase, a transferase, a hydrolase, a lyase, an isomerase, or a ligase.

[32] The mutant of any of paragraphs 1-31, wherein the parent bacterial cell is a Bacillus cell.

[33] The mutant of paragraph 32, wherein the Bacillus cell is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cell.

[34] The mutant of paragraph 32, wherein the Bacillus cell is a Bacillus subtilis cell.

[35] The mutant of paragraph 32, wherein the Bacillus cell is a Bacillus licheniformis cell.

[36] The mutant of any of paragraphs 1-35, which produces at least about 25% less of the thiol-disulfide oxidoreductase compared to the parent bacterial cell when cultured under identical conditions.

[37] The mutant of any of paragraphs 1-35, which produces no detectable thiol-disulfide oxidoreductase compared to the parent bacterial cell when cultured under identical conditions.

[38] A method of producing a heterologous polypeptide, comprising: (a) cultivating a mutant of a parent bacterial cell in a medium for the production of the heterologous polypeptide, wherein (i) the mutant cell comprises a first polynucleotide encoding the heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide, and (ii) the mutant cell is deficient in production of the thiol-disulfide oxidoreductase compared to the parent bacterial cell when cultivated under the same conditions; and (b) recovering the heterologous polypeptide from the cultivation medium.

[39] The method of paragraph 38, wherein the thiol-disulfide oxidoreductase gene is selected from the group consisting of: (a) a gene encoding a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof; (b) a gene encoding a thiol-disulfide oxidoreductase comprising a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; and (c) a gene encoding a thiol-disulfide oxidoreductase comprising a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[40] The method of paragraph 39, wherein the thiol-disulfide oxidoreductase gene encodes a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[41] The method of paragraph 40, wherein the thiol-disulfide oxidoreductase gene encodes a thiol-disulfide oxidoreductase comprising an amino acid sequence having at least 65% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[42] The method of paragraph 41, wherein the thiol-disulfide oxidoreductase gene encodes a thiol-disulfide oxidoreductase comprising an amino acid sequence having at least 70% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[43] The method of paragraph 42, wherein the thiol-disulfide oxidoreductase gene encodes a thiol-disulfide oxidoreductase comprising an amino acid sequence having at least 75% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[44] The method of paragraph 43, wherein the thiol-disulfide oxidoreductase gene encodes a thiol-disulfide oxidoreductase comprising an amino acid sequence having at least 80% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[45] The method of paragraph 44, wherein the thiol-disulfide oxidoreductase gene encodes a thiol-disulfide oxidoreductase comprising an amino acid sequence having at least 85% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[46] The method of paragraph 45, wherein the thiol-disulfide oxidoreductase gene encodes a thiol-disulfide oxidoreductase comprising an amino acid sequence having at least 90% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[47] The method of paragraph 46, wherein the thiol-disulfide oxidoreductase gene encodes a thiol-disulfide oxidoreductase comprising an amino acid sequence having at least 95% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[48] The method of paragraph 47, wherein the thiol-disulfide oxidoreductase gene encodes a thiol-disulfide oxidoreductase comprising an amino acid sequence having at least 97% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[49] The method of paragraph 48, wherein the thiol-disulfide oxidoreductase gene encodes a thiol-disulfide oxidoreductase comprising an amino acid sequence having at least 99% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[50] The method of paragraph 39, wherein the thiol-disulfide oxidoreductase gene comprises a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[51] The method of paragraph 50, wherein the thiol-disulfide oxidoreductase gene comprises a nucleotide sequence that hybridizes under at least medium stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[52] The method of paragraph 51, wherein the thiol-disulfide oxidoreductase gene comprises a nucleotide sequence that hybridizes under at least medium-high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[53] The method of paragraph 52, wherein the thiol-disulfide oxidoreductase gene comprises a nucleotide sequence that hybridizes under at least high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[54] The method of paragraph 53, wherein the thiol-disulfide oxidoreductase gene comprises a nucleotide sequence that hybridizes under at least very high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[55] The method of paragraph 39, wherein the thiol-disulfide oxidoreductase gene comprises a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[56] The method of paragraph 55, wherein the thiol-disulfide oxidoreductase gene comprises a nucleotide sequence having at least 65% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[57] The method of paragraph 56, wherein the thiol-disulfide oxidoreductase gene comprises a nucleotide sequence having at least 70% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[58] The method of paragraph 57, wherein the thiol-disulfide oxidoreductase gene comprises a nucleotide sequence having at least 75% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[59] The method of paragraph 58, wherein the thiol-disulfide oxidoreductase gene comprises a nucleotide sequence having at least 80% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[60] The method of paragraph 59, wherein the thiol-disulfide oxidoreductase gene comprises a nucleotide sequence having at least 85% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[61] The method of paragraph 60, wherein the thiol-disulfide oxidoreductase gene comprises a nucleotide sequence having at least 90% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[62] The method of paragraph 61, wherein the thiol-disulfide oxidoreductase gene comprises a nucleotide sequence having at least 95% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[63] The method of paragraph 62, wherein the thiol-disulfide oxidoreductase gene comprises a nucleotide sequence having at least 97% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[64] The method of paragraph 63, wherein the thiol-disulfide oxidoreductase gene comprises a nucleotide sequence having at least 99% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[65] The method of paragraph 38, wherein the thiol-disulfide oxidoreductase gene encodes the thiol-disulfide oxidoreductase of SEQ ID NO: 2, SEQ ID NO: 50, SEQ ID NO: 52, SEQ ID NO: 54, SEQ ID NO: 56, SEQ ID NO: 58, or SEQ ID NO: 60.

[66] The method of paragraph 38, wherein the thiol-disulfide oxidoreductase gene encodes the thiol-disulfide oxidoreductase of SEQ ID NO: 4, SEQ ID NO: 62, SEQ ID NO: 64, SEQ ID NO: 66, or SEQ ID NO: 68.

[67] The method of any of paragraphs 38-66, wherein the polypeptide encoded by the first polynucleotide is an antigen, an enzyme, a growth factor, a hormone, an immunodilator, a neurotransmitter, a receptor, a reporter protein, a structural protein, or a transcription factor.

[68] The method of paragraph 67, wherein the enzyme is an oxidoreductase, a transferase, a hydrolase, a lyase, an isomerase, or a ligase.

[69] The method of any of paragraphs 38-68, wherein the parent bacterial cell is a Bacillus cell.

[70] The method of paragraph 69, wherein the Bacillus cell is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cell.

[71] The method of paragraph 69, wherein the Bacillus cell is a Bacillus subtilis cell.

[72] The method of paragraph 69, wherein the Bacillus cell is a Bacillus licheniformis cell.

[73] The method of any of paragraphs 38-72, wherein the mutant cell produces at least about 25% less of the thiol-disulfide oxidoreductase compared to the parent bacterial cell when cultured under identical conditions.

[74] The method of any of paragraphs 38-72, wherein the mutant cell produces no detectable thiol-disulfide oxidoreductase compared to the parent bacterial cell when cultured under identical conditions.

[75] A method of obtaining a mutant of a parent bacterial cell, comprising: (a) introducing into the parent bacterial cell a first polynucleotide encoding a heterologous polypeptide which comprises two or more (several) cysteines, and a second polynucleotide comprising a modification of a gene encoding a thiol-disulfide oxidoreductase that incorrectly catalyzes the formation of one or more (several) disulfide bonds between the two or more (several) cysteines of the heterologous polypeptide; and (b) identifying the mutant cell from step (a) comprising the modified polynucleotide, wherein the mutant cell is deficient in the production of the thiol-disulfide oxidoreductase.

[76] The method of paragraph 75, wherein the thiol-disulfide oxidoreductase gene is selected from the group consisting of: (a) a gene encoding a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof; (b) a gene encoding a thiol-disulfide oxidoreductase comprising a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; and (c) a gene encoding a thiol-disulfide oxidoreductase comprising a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[77] The method of paragraph 76, wherein the thiol-disulfide oxidoreductase gene encodes a thiol-disulfide oxidoreductase comprising an amino acid sequence having at least 60% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[78] The method of paragraph 77, wherein the thiol-disulfide oxidoreductase gene encodes a thiol-disulfide oxidoreductase comprising an amino acid sequence having at least 65% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[79] The method of paragraph 78, wherein the thiol-disulfide oxidoreductase gene encodes a thiol-disulfide oxidoreductase comprising an amino acid sequence having at least 70% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[80] The method of paragraph 79, wherein the thiol-disulfide oxidoreductase gene encodes a thiol-disulfide oxidoreductase comprising an amino acid sequence having at least 75% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[81] The method of paragraph 80, wherein the thiol-disulfide oxidoreductase gene encodes a thiol-disulfide oxidoreductase comprising an amino acid sequence having at least 80% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[82] The method of paragraph 81, wherein the thiol-disulfide oxidoreductase gene encodes a thiol-disulfide oxidoreductase comprising an amino acid sequence having at least 85% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[83] The method of paragraph 82, wherein the thiol-disulfide oxidoreductase gene encodes a thiol-disulfide oxidoreductase comprising an amino acid sequence having at least 90% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[84] The method of paragraph 83, wherein the thiol-disulfide oxidoreductase gene encodes a thiol-disulfide oxidoreductase comprising an amino acid sequence having at least 95% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[85] The method of paragraph 84, wherein the thiol-disulfide oxidoreductase gene encodes a thiol-disulfide oxidoreductase comprising an amino acid sequence having at least 97% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[86] The method of paragraph 85, wherein the thiol-disulfide oxidoreductase gene encodes a thiol-disulfide oxidoreductase comprising an amino acid sequence having at least 99% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof.

[87] The method of paragraph 76, wherein the thiol-disulfide oxidoreductase gene comprises a nucleotide sequence that hybridizes under at least low stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[88] The method of paragraph 87, wherein the thiol-disulfide oxidoreductase gene comprises a nucleotide sequence that hybridizes under at least medium stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[89] The method of paragraph 88, wherein the thiol-disulfide oxidoreductase gene comprises a nucleotide sequence that hybridizes under at least medium-high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[90] The method of paragraph 89, wherein the thiol-disulfide oxidoreductase gene comprises a nucleotide sequence that hybridizes under at least high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[91] The method of paragraph 90, wherein the thiol-disulfide oxidoreductase gene comprises a nucleotide sequence that hybridizes under at least very high stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof.

[92] The method of paragraph 76, wherein the thiol-disulfide oxidoreductase gene comprises a nucleotide sequence having at least 60% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[93] The method of paragraph 92, wherein the thiol-disulfide oxidoreductase gene comprises a nucleotide sequence having at least 65% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[94] The method of paragraph 93, wherein the thiol-disulfide oxidoreductase gene comprises a nucleotide sequence having at least 70% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[95] The method of paragraph 94, wherein the thiol-disulfide oxidoreductase gene comprises a nucleotide sequence having at least 75% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[96] The method of paragraph 95, wherein the thiol-disulfide oxidoreductase gene comprises a nucleotide sequence having at least 80% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[97] The method of paragraph 96, wherein the thiol-disulfide oxidoreductase gene comprises a nucleotide sequence having at least 85% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[98] The method of paragraph 97, wherein the thiol-disulfide oxidoreductase gene comprises a nucleotide sequence having at least 90% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[99] The method of paragraph 98, wherein the thiol-disulfide oxidoreductase gene comprises a nucleotide sequence having at least 95% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[100] The method of paragraph 99, wherein the thiol-disulfide oxidoreductase gene comprises a nucleotide sequence having at least 97% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[101] The method of paragraph 100, wherein the thiol-disulfide oxidoreductase gene comprises a nucleotide sequence having at least 97% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

[102] The method of paragraph 75, wherein the thiol-disulfide oxidoreductase gene encodes the thiol-disulfide oxidoreductase of SEQ ID NO: 2, SEQ ID NO: 50, SEQ ID NO: 52, SEQ ID NO: 54, SEQ ID NO: 56, SEQ ID NO: 58, or SEQ ID NO: 60.

[103] The method of paragraph 75, wherein the thiol-disulfide oxidoreductase gene encodes the thiol-disulfide oxidoreductase of SEQ ID NO: 4, SEQ ID NO: 62, SEQ ID NO: 64, SEQ ID NO: 66, or SEQ ID NO: 68.

[104] The method of any of paragraphs 75-103, wherein the polypeptide encoded by the first polynucleotide is an antigen, an enzyme, a growth factor, a hormone, an immunodilator, a neurotransmitter, a receptor, a reporter protein, a structural protein, or a transcription factor.

[105] The method of paragraph 104, wherein the enzyme is an oxidoreductase, a transferase, a hydrolase, a lyase, an isomerase, or a ligase.

[106] The method of any of paragraphs 75-105, wherein the parent bacterial cell is a Bacillus cell.

[107] The method of paragraph 106, wherein the Bacillus cell is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cell.

[108] The method of paragraph 106, wherein the Bacillus cell is a Bacillus subtilis cell.

[109] The method of paragraph 106, wherein the Bacillus cell is a Bacillus licheniformis cell.

[110] The method of any of paragraphs 75-109, wherein the mutant cell produces at least about 25% less of the thiol-disulfide oxidoreductase compared to the parent bacterial cell when cultured under identical conditions.

[111] The method of any of paragraphs 75-109, wherein the mutant cell produces no detectable thiol-disulfide oxidoreductase compared to the parent bacterial cell when cultured under identical conditions.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. An isolated mutant of a parent bacterial cell, comprising a first polynucleotide encoding a heterologous polypeptide which comprises two or more cysteines, and a second polynucleotide comprising a modification of a gene encoding a thiol-disulfide oxidoreductase that incorrectly catalyzes the formation of one or more disulfide bonds between the two or more cysteines of the heterologous polypeptide, wherein the mutant cell is deficient in production of the thiol-disulfide oxidoreductase compared to the parent bacterial cell when cultivated under the same conditions.

2. The mutant of claim 1, wherein the thiol-disulfide oxidoreductase gene is selected from the group consisting of:
(a) a gene encoding a thiol-disulfide oxidoreductase comprising an amino acid sequence having at least 70% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof;
(b) a gene encoding a thiol-disulfide oxidoreductase comprising a nucleotide sequence that hybridizes under medium stringency conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; and
(c) a gene encoding a thiol-disulfide oxidoreductase comprising a nucleotide sequence having at least 70% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

3. The mutant of claim 1, wherein the polypeptide encoded by the first polynucleotide is an antigen, an enzyme, a growth factor, a hormone, an immunodilator, a neurotransmitter, a receptor, a reporter protein, a structural protein, or a transcription factor.

4. The mutant of claim 1, wherein the parent bacterial cell is a Bacillus cell.

5. The mutant of claim 4, wherein the Bacillus cell is a Bacillus subtilis cell or a Bacillus licheniformis cell.

6. The mutant of claim 1, which produces no detectable or at least about 25% less of the thiol-disulfide oxidoreductase compared to the parent bacterial cell when cultured under identical conditions.

7. A method of producing a heterologous polypeptide, comprising:
(a) cultivating the mutant of claim 1 in a medium for the production of the heterologous polypeptide; and
(b) recovering the heterologous polypeptide from the cultivation medium.

8. A method of obtaining a mutant of a parent bacterial cell, comprising:
(a) introducing into the parent bacterial cell a first polynucleotide encoding a heterologous polypeptide which comprises two or more cysteines, and a second polynucleotide comprising a modification of a gene encoding a thiol-disulfide oxidoreductase that incorrectly catalyzes the formation of one or more (several) disulfide bonds between the two or more cysteines of the heterologous polypeptide; and
(b) identifying the mutant cell from step (a) comprising the modified polynucleotide, wherein the mutant cell is deficient in the production of the thiol-disulfide oxidoreductase.

9. The method of claim 8, wherein the thiol-disulfide oxidoreductase gene is selected from the group consisting of:
(a) a gene encoding a thiol-disulfide oxidoreductase comprising an amino acid sequence having at least 70% sequence identity to SEQ ID NO: 2, SEQ ID NO: 4, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, or SEQ ID NO: 68; or the mature polypeptide thereof;
(b) a gene encoding a thiol-disulfide oxidoreductase comprising a nucleotide sequence that hybridizes under medium stringency conditions, conditions with SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; the mature polypeptide coding sequence thereof; or the full-length complementary strand thereof; and
(c) a gene encoding a thiol-disulfide oxidoreductase comprising a nucleotide sequence having at least 70% sequence identity to SEQ ID NO: 1, SEQ ID NO: 3, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, or SEQ ID NO: 67; or the mature polypeptide coding sequence thereof.

10. The method of claim 8, wherein the polypeptide encoded by the first polynucleotide is an antigen, an enzyme, a growth factor, a hormone, an immunodilator, a neurotransmitter, a receptor, a reporter protein, a structural protein, or a transcription factor.

11. The method of claim 8, wherein the parent bacterial cell is a Bacillus cell.

12. The method of claim 11, wherein the Bacillus cell is a Bacillus subtilis cell or a Bacillus licheniformis cell.

13. The method of claim 8, wherein the mutant cell produces no detectable or at least about 25% less of the thiol-disulfide oxidoreductase compared to the parent bacterial cell when cultured under identical conditions.
